(19) United States
(12) Patent Application Publication
(10) Pub. No.: US 2026/0324650 A1
(43) Pub. Date: Oct. 22, 2026

(54) Derivatives of Bacillus Strains for Inhibition of Plant Disease

(71) Applicant: CHR. HANSEN A/S, Hoersholm (DK)

(72) Inventors: Sailesh MALLA, Hoersholm (DK); Ronnie MACHIELSEN, Hoersholm (DK); Patricia Dominguez CUEVAS, Hoersholm (DK); Edward ROJAS, Hoersholm (DK); Miguel TOVAR, Hoersholm (DK)

(73) Assignee: CHR. HANSEN A/S, Hoersholm (DK)

(21) Appl. No.: 19/104,395

(22) PCT Filed: Aug. 28, 2023
(86) PCT No.: PCT/EP2023/073504

§ 371 Date: Feb. 18, 2025

(30) Foreign Application Priority Data
Aug. 30, 2022 (EP) 22192737.9

(51) Int. Cl. A01N 63/22; A01P 3/00; C12N 1/205; C12R 1/07

(52) U.S. Cl. CPC A01N 63/22; A01P 3/00; C12N 1/205; C12R 2001/07

(57) ABSTRACT

The present invention relates to provision of bacteria for growth inhibition of phytopathogens. In particular, the present invention relates to provision of Bacillus amyloliquefaciens derivative strains capable of inhibiting growth of phytopathogens such as Fusarium graminearum and/or Botrytis cinerea.

### TECHNICAL FIELD OF THE INVENTION

The present invention relates to provision of bacteria for inhibition of plant disease. In particular, the present invention relates to provision of derivative strains of Bacillus amyloliquefaciens capable of inhibiting growth of phytopathogens, such as Fusarium graminearum and Botrytis cinerea.

### BACKGROUND OF THE INVENTION

The annual global production of four major crops; maize, wheat, rice and barley, are ca. 1110 million metric tons, ca. 750 million metric tons, ca. 500 million metric tons and ca. 150 million metric tons, respectively. The rapid population growth combined with climate change create a big challenge for crop production and yield globally. On one hand, there is an increasing demand of agricultural yield while on the other hand various biotic and abiotic issues significantly reduce crop production.

Among the plant diseases, fungal pathogens are the biggest global threat causing huge losses in agriculture and food production. Fungal pathogens, Fusarium graminearum and Fusarium culmorum and other Fusarium spp., cause fusarium head blight (FHB), which is a devastating crop disease on maize, wheat, rice and barley causing billions of dollars in economic losses worldwide annually. These fungi contaminate seeds of the above-mentioned crops mainly with two types of mycotoxins; trichothecene deoxynivalenol and zearalenone, which are produced during infection. Furthermore, both mycotoxins have hazardous effects on human and animal health. The Fusarium spp. fungi is often present in soils where maize has been cultivated and can remain in the soil for several seasons. FHB symptoms are seen as discoloration or yellowing of glumes and spikelets after flowering.

Various strategies have been implemented to inhibit and control phytopathogens, such as Fusarium graminearum and Fusarium culmorum, including application of chemical fungicides, crop rotation, seed treatment etc. Even though the correct usage of fungicide at an early heading date can reduce a disease such as FHB by 50-60%, application of fungicides is challenging due to overlapping of different developmental stages within the crop. FHB disease generally develops late in the season or also during storage of the crops/seeds indicating that early application of fungicides might only be partially effective. Furthermore, prolonged use of chemically synthesized fungicides reduce microbial biodiversity in soil, increases pathogen resistance and generally degrades the soil quality.

Botrytis cinerea is an airborne fungal pathogen causing gray mold in flowers and fruits of plants, particularly at the end of the flowering or fruit-ripening period and is a devastating disease for fruits, vegetables and ornamental crops. The fungi can overwinter on crop debris where spring temperatures and rain initiate the sporulation process. These spores are transported by wind and rain to the flowers and fruits of host plants. Usual symptoms include spotting of fruits, discoloration and rotting of fruits. Additionally, the fungus rapidly sporulates over infected tissue creating a black or gray velvety mold and spreading to neighboring plants. Gray mold is the most serious of all fungal diseases and repeated application of fungicide is necessary to control the disease, especially in mild and humid climates. Due to limited broad-spectrum active ingredients, chemical control of Botrytis cinerea highly relies on fungicides with specific modes of action. This is pushing the development of Botrytis populations with low sensitivity to fungicides. in greenhouses and fields. Therefore, there is an increasing need for improved and sustainable solutions for controlling the occurrence of Botrytis cinerea in plants.

As evident from the above, the conventional treatment and control of FBH and gray mold requires different strategies and compositions in order to be fully effective.

It is therefore desirable to develop products and control and/or treatment methodologies that can easily be implemented in standard agriculture for combatting these phytopathogens, preferentially with one broad-range product.

Plant and soil microbes interact to help each other for their growth and development as well as to maintain the terrestrial eco-system. Plants can also use these growth promoting microbes as weapon against various phytopathogens including fungi as microbes have great potential to produce and secrete various bioactive molecules that can act against the phytopathogens, such as fungal pathogens.

Growth promoting microbes include Bacillus which are Gram-positive bacteria characterized by having thick cell walls and the absence of outer membranes. Much of the cell wall of Gram-positive bacteria is composed of peptidoglycan. Gram-positive species are divided into groups according to their morphological and biochemical characteristics. The genus Bacillus is belonging to the group of sporulating bacteria. Bacterial spores are one of the most resilient cell types; they resist many environmental changes, withstand dry heat and certain chemical disinfectants and may persist for years on dry land.

Accordingly, Bacillus industrial strains are routinely applied in various plant health products for plantations. Many of these industrial Bacillus strains produce/secrete various classes of bioactive metabolites, for example non-ribosomal polyketide synthases (NRPS), polyketides, siderophores, antibiotics, surfactant, hydrolytic enzymes (e.g., protease, lipase, etc.), volatile compounds, etc.

Lipopeptides (e.g. surfactins, iturins, fengycins and the like) are bioactive metabolites produced by Bacillus strains capable of inhibiting growth of phytopathogens, e.g. as working as fungicides against FHB and gray mold. However, the available industrial Bacillus strains have potential to be further developed/improved for combating phytopathogens, including those causing fungal diseases such as FHB and gray mold. Improving the efficiency of biostimulant Bacillus strains would come with significant economic savings and improve the ability to meet the increasing global demands for crop production as the world population grow.

Furthermore, besides the direct antagonism mechanisms related to the various classes of bioactive metabolites produced by Bacillus strains, some beneficial bacteria can protect plants indirectly through the stimulation of the plant defense mechanisms, also known as induced systemic resistance (ISR). Elicitation of the ISR renders plants more resistant to pathogen infection. Protection resulting from ISR elicited by Bacillus spp. has been reported against both fungal and bacterial pathogens. It is contemplated that the Bacillus strains, or compositions containing those, as described herein are able to also stimulate ISR in the plants after having been applied to the plants or the habitat, preferably applied to the foliage.

Thus, there is an unmet need for supplying improved strains of Bacillus to combat phytopathogens and methods for their use to improve plant health and yield of crops without utilizing hazardous chemical fungicides.

Hence, it would be advantageous to provide improved Bacillus strains capable of inhibiting phytopathogens, such as Fusarium graminearum, Fusarium culmorum and Botrytis cinerea. Specifically, it would be advantageous to provide Bacillus strains with enhanced production of bioactive (antifungal) metabolites, such as lipopeptides, that may be utilized as efficient and climate friendly solutions for improving crop yield with activity against Fusarium spp. and Botrytis spp.

### SUMMARY OF THE INVENTION

The present invention relates to Bacillus strains that are effective in inhibiting phytopathogens. In particular, the present invention discloses derivatives of parental Bacillus strains that may be applied to plants or the habitat of the plant to improve plant health and growth. The derivative Bacillus strains are prepared through natural methods of classical strain improvement by exposure of a parental Bacillus strain to a mutagen followed by identification and selection of improved strains. Accordingly, the present invention makes available improved Bacilli to combat phytopathogens, such as Fusarium graminearum, Fusarium culmorum and Botrytis cinerea, reducing the need for chemical fungicides.

Thus, an object of the present invention relates to the provision of improved Bacillus strains capable of inhibiting plant disease in a climate friendly manner.

In particular, it is an object of the present invention to provide derivative strains of Bacillus amyloliquefaciens that effectively protects plants against fungal pathogens, such as Fusarium graminearum, Fusarium culmorum and Botrytis cinerea, thereby lessening the loss of crops to disease.

Thus, an aspect of the present invention relates to a Bacillus amyloliquefaciens strain, or a mutant or variant thereof, with increased inhibitory effect on one or more phytopathogens compared to a parental strain of Bacillus amyloliquefaciens.

Another aspect of the present invention relates to a Bacillus amyloliquefaciens strain, or a mutant or variant thereof, with increased inhibitory effect on one or more plant fungal pathogens or plant bacterial pathogens compared to a parental strain of Bacillus amyloliquefaciens (FHB-P) deposited as DSM34003 at Deutsche Sammlung von Mikroorganismen und Zellkulturen GmbH (DSMZ), Inhoffenstr. 7B, D-38124 Braunschweig, Germany, by Chr. Hansen A/S, Hørsholm, Denmark on 24 Aug. 2021, wherein the Bacillus amyloliquefaciens strain, or a mutant or variant thereof, is a derivative strain of FHB-P.

Yet another aspect of the present invention relates to a method for preparing a Bacillus amyloliquefaciens strain as described herein, said method comprising the steps of:
(i) providing a parental strain of Bacillus amyloliquefaciens;
(ii) subjecting said parental strain of Bacillus amyloliquefaciens to one or more mutagens to generate one or more Bacillus amyloliquefaciens strains;
(iii) determine the inhibitory effect and/or production of one or more metabolites of said one or more Bacillus amyloliquefaciens strains; and
(iv) selecting a Bacillus amyloliquefaciens strain with increased inhibitory effect and/or increased production of one or more metabolites.

Still another aspect of the present invention relates to a Bacillus amyloliquefaciens strain obtainable by the method as described herein.

A further aspect of the present invention relates to a composition comprising a Bacillus amyloliquefaciens strain as described herein.

A still further aspect of the present invention relates to a method of inhibiting growth of one or more phytopathogens on a plant, said method comprising applying a Bacillus amyloliquefaciens strain or a composition as described herein to the plant or a part of the plant.

An even further aspect of the present invention relates to use of a Bacillus amyloliquefaciens strain or a composition as described herein for inhibiting growth of one or more phytopathogens on a plant.

Yet another aspect of the present invention relates to a kit comprising:
(i) a Bacillus amyloliquefaciens strain or a composition as described herein;
(ii) a container; and
(iii) optionally, instructions for use.

### BRIEF DESCRIPTION OF THE FIGURES

FIG. 1 shows evaluation of optimal ethyl methane sulphonate (EMS) concentration. A) Effect of EMS in Bacillus growth. Various concentrations (0-91 g/L) of EMS were added in the growth media (LB) to cultivate Bacillus amyloliquefaciens FHB-P. B) CFU/ml of Bacillus amyloliquefaciens FHB-P with (solid line) and without (dotted line) EMS treatment (18 g/L) over the time. C) Determination of killing rate percentage of Bacillus amyloliquefaciens FHB-P by EMS (18 g/L) based on CFU/ml.

FIG. 2 shows spore germination and growth of Fusarium graminearum in 10⁻¹ to 10⁻⁸ fold serially diluted spore stocks in PDB+Cm media. Left: OD600 measurement values of the microtiter plate. Right: Images of the microtiter plate growing fungi. Along the row is 10-fold decrease in spore stock and along the column (from A to F) are replicates.

FIG. 3 shows the Fusarium inhibition assay for screening isolates from EMS libraries of Bacillus amyloliquefaciens FHB-P. The first and second plates are supplemented with 7.5 μl and 10 μl of saturated Bacillus fermentation products, respectively. Wells A1, B1 and C1 (dark rectangle) are supplemented with parental Bacillus amyloliquefaciens FHB-P. The selected hits are shown in white dotted circles, where Fusarium growth is markedly reduced as compared to the corresponding parental control.

FIG. 4 shows the relative improvement of Fusarium inhibition by fermentation product from thirteen selected lead Bacillus strains. Empty refers to supplementation of LB medium instead of Bacillus fermentation product.

FIG. 5 shows sporulation test of the selected leads from the manual screening. A) Observation under microscope for sporulating capacity of the selected leads. All of the lead strains, except FHB-S5, are spore positive.

FIG. 6 shows production of lipopeptides (iturin, fengycin, surfactin) of the lead strains selected by manual (A) and automated (B) screening. Assay was done in LB media. FHB-P is the parental strain of Bacillus amyloliquefaciens.

FIG. 7 shows production of lipopeptides (iturin, fengycin and surfactin) of lead strains and the parental Bacillus amyloliquefaciens FHB-P strain after 48 h of incubation at 37 °C and 250 RPM. Assay was done in MSgg media.

FIG. 8 shows determination of Fusarium inhibition activity of derivative strains of Bacillus amyloliquefaciens in (A) LB media or (B) MSgg media. The inhibition assay was carried out by supplementing 5, 7.5, 10 and 13 μl of the Bacillus fermentation products, from bacterial cultures grown in LB media or MSgg media, in the PDB+Cm media with Fusarium spores. The Fusarium growth in presence of the Bacillus parental strain fermentation product is shown as black bars. All the derivative strains to the left side of the black bars have improved Fusarium inhibition activity as compared to the parental strain.

FIG. 9 shows biofungicide potency of selected improved derivative strains and their parental Bacillus amyloliquefaciens strain against the phytopathogenic filamentous fungi Fusarium graminearum. (A) Fungal growth inhibition potency of the fermentation products of derivative strains is displayed as fold change of ID50 values normalised to the ID50 value of the parental Bacillus amyloliquefaciens strain. (B) Corresponding levels of bioactive metabolites belonging to the three main lipopeptide (LP) families (iturins, fengycins, surfactins) produced by the derivative strains displayed as fold change normalised to the LP concentrations of the parental Bacillus amyloliquefaciens strain. Data corresponds to averages from 2-3 biologically independent experiments. Error bars correspond to the standard deviation values between the samples. Assay was done in LC, Chr. Hansen production medium.

FIG. 10 shows biofungicide potency of selected improved derivative strains and their parental Bacillus amyloliquefaciens strain against the phytopathogenic filamentous fungi Botrytis cinerea. (A) Fungal growth inhibition potency of the fermentation products of derivative strains is displayed as fold change of ID50 values normalised to the ID50 value of the parental Bacillus amyloliquefaciens strain. (B) Corresponding levels of bioactive metabolites belonging to the three main lipopeptide (LP) families (iturins, fengycins, surfactins) produced by the derivative strains displayed as fold change normalised to the LP concentrations of the parental Bacillus amyloliquefaciens strain. Data corresponds to averages from 2-3 biologically independent experiments. Error bars correspond to the standard deviation values between the samples. Assay was done in M2 medium.

The present invention will in the following be described in more detail.

### DETAILED DESCRIPTION OF THE INVENTION

### Definitions

Prior to outlining the present invention in more details, a set of terms and conventions is first defined:

### Plant Biostimulant

In the present context, the term “plant biostimulant” refers to any substance or microorganism applied to plants with the ability to enhance nutrition efficiency, abiotic stress tolerance and/or crop quality traits, regardless of its nutrients content. By extension, plant biostimulants also designate commercial products containing mixtures of such substances and/or microorganisms.

### Plant Growth Promoting Agent

In the present context, the term “plant growth promoting agent” or “plant growth promoting microorganism” refers to a microorganism with the ability to colonize roots, aerial plant surfaces (leaves, stems, flowers, fruits) and/or inner plant tissues and promote plant growth and health by either acting as a biofertilizer, biostimulant, biocontrol agent, or via biological control of plant disease.

### Phytopathogens

In the present context, the term “phytopathogen” refers to any microorganism that is pathogenic to plants. In particular, phytopathogens include, but are not limited to, fungi and bacteria.

### Inhibitory Effect

In the present context, the term “inhibitory effect” refers to the ability of a microorganism to kill or reduce the growth of a phytopathogen. Accordingly, the inhibitory effect may be determined by quantifying the amount of the phytopathogen upon exposure to the microorganism.

### Parental Strain

In the present context, the term “parental strain” refers to a microorganism that is the origin to one or more derived strains, i.e. it is designating the first generation giving rise to one or more succeeding generations.

### Derivative Strain

In the present context, the term “derivative strain” refers to a microorganism that is a second generation derived from a parental strain. The derivative strain may be developed by mutagenesis, wherein one or more mutations are incorporated into the genome of the parental strain. The mutations may be acquired by induced mutagenesis by exposing the parental strain to one or more mutagens. The mutagens may be chemical mutagens or physical mutagens.

### Mutagen

In the present context, the term “mutagen” refers to any physical or chemical agent that increases the frequency of mutations in the genetic material in an organism above the natural background level of mutations. The frequency of mutations and mode of action may vary depending on the mutagen. Preferably, the mutagen primarily modifies the DNA sequence by substitution, insertion or deletion of one or more nucleotides in the DNA sequence.

### Identifying Characteristics

In the present context, the term “identifying characteristics” refers to the phenotype of a microorganism, i.e. the set of observable characteristics or traits of the microorganism. Particularly, the identifying characteristic can be the inhibitory effect on a phytopathogen and/or production of one or more metabolites. The metabolite may be a lipopeptide, such as compounds belonging to the iturins, fengycins and/or surfactins families.

Microorganisms sharing all identifying characteristics can have different non-identical genomic sequences. This may be the case if mutations are silent or conservative, i.e. the new codon gives rise to the same amino acid or the new amino acid have similar biochemical properties (e.g. charge or hydrophobicity), respectively.

### Mutant or Mutant Strain

In the present context, the term “mutant” or “mutant strain” refers to a strain derived, or a strain which can be derived, from a strain of the invention (or the parental strain) by means of e.g. genetic engineering, mutagenesis, radiation and/or chemical treatment. It is preferred that the mutant is a functionally equivalent mutant, e.g. a mutant that has substantially the same, or improved, properties (e.g. regarding the inhibitory ability against phytopathogens) as the strain from which it is derived. Such a mutant is a part of the present invention.

Especially, the term “mutant” refers to a strain obtained by subjecting a strain of the invention to any conventionally used mutagenesis treatment including classical strain improvement, treatment with a chemical mutagen such as ethane methane sulphonate (EMS) or N-methyl-N′-nitro-N-nitroguanidine (NTG), UV light, or to a spontaneously occurring mutant. A mutant may have been subjected to several mutagenesis treatments (a single treatment should be understood as one mutagenesis step followed by a screening/selection step), but it is presently preferred that no more than 20, or no more than 10, or no more than 5, treatments (or screening/selection steps) are carried out.

In a presently preferred mutant, less than 5%, or less than 1% or even less than 0.1% of the nucleotides in the bacterial genome have been modified by replacement with another nucleotide, or deleted, compared to the parental strain. As will be clear to the skilled person mutants of the present invention can also be parental strains.

### Nomenclature of Mutation

In the present context, the conventional one-letter and three-letter codes for amino acid residues are used. For ease of reference, amino acid changes in mutants and variants of the invention are described by use of the following nomenclature: amino acid residue in the parent enzyme; position; substituted amino acid residue(s).

According to this nomenclature, the substitution of, for instance, an alanine residue for a glycine residue at position 20 is indicated as Ala20Gly or A20G. The deletion of alanine in the same position is shown as Ala20* or A20*. The insertion of an additional amino acid residue (e.g. a glycine) is indicated as Ala20AlaGly or A20AG. The deletion of a consecutive stretch of amino acid residues (e.g. between alanine at position 20 and glycine at position 21) is indicated as DELTA(Ala20-Gly21) or DELTA(A20-G21). When a parent enzyme sequence contains a deletion in comparison to the enzyme sequence used for numbering an insertion in such a position (e.g. an alanine in the deleted position 20) is indicated as *20Ala or *20A. Multiple mutations are separated by a plus sign or a slash. For example, two mutations in positions 20 and 21 substituting alanine and glutamic acid for glycine and serine, respectively, are indicated as A20G+E21S or A20G/E21S. When an amino acid residue at a given position is substituted with two or more alternative amino acid residues these residues are separated by a comma or a slash. For example, substitution of alanine at position 30 with either glycine or glutamic acid is indicated as A20G,E or A20G/E, or A20G, A20E.

When a position suitable for modification is identified herein without any specific modification being suggested, it is to be understood that any amino acid residue may be substituted for the amino acid residue present in the position. Thus, for instance, when a modification of an alanine in position 20 is mentioned but not specified, it is to be understood that the alanine may be deleted or substituted for any other amino acid residue (i.e. any one of R, N, D, C, Q, E, G, H, I, L, K, M, F, P, S, T, W, Y, V).

### Mutation

In the present context, the term “mutation” refers to an alteration in the nucleotide sequence of the genome of an organism resulting in changes in the phenotype of said organism, wherein the alteration may be a deletion of a nucleotide, a substitution of a nucleotide by another nucleotide, an insertion of a nucleotide, or a frameshift.

A deletion is to be understood as a genetic mutation resulting in the removal of one or more nucleotides of a nucleotide sequence of the genome of an organism; an insertion is to be understood as the addition of one or more nucleotides to the nucleotide sequence; a substitution (or point mutation) is to be understood as a genetic mutation where a nucleotide of a nucleotide sequence is substituted by another nucleotide; a frameshift is to be understood as a genetic mutation caused by a insertion or deletion of a number of nucleotides in a nucleotide sequence that is not divisible by three, therefore changing the reading frame and resulting in a completely different translation from the original reading frame; an introduction of a stop codon is to be understood as a point mutation in the DNA sequence resulting in a premature stop codon; an inhibition of substrate binding of the encoded protein is to be understood as any mutation in the nucleotide sequence that leads to a change in the protein sequence responsible for preventing binding of a substrate to its catalytic site of the protein.

Furthermore, a knockout mutant is to be understood as genetic mutation resulting in the removal or deletion of a gene, such as an entire gene or an entire open reading frame from the genome of an organism.

In the present description and claims the conventional one-letter code for nucleotides is used following the analogous principles as described for amino acids nomenclature supra.

### Variant or Variant Strain

In the present context, the term “variant” or “variant strain” refers to a strain which is functionally equivalent to a strain of the invention, e.g. having substantially the same properties (e.g. regarding the inhibitory ability against phytopathogens). Such variants, which may be identified using appropriate screening techniques, are a part of the present invention.

### Fermentation Product

In the present context, the term “fermentation product” refers to the bacterial culture containing media components, compounds secreted by the bacterial cells resulting from metabolism, such as lipopeptides, polyketides and enzymes, and products from transformations of compounds present in the media or secreted by the bacterial cells. The fermentation product may also contain bacterial cells, in the vegetative and/or spore form and cell debris.

### Metabolite

In the present context, the term “metabolite” refers to any substance produced as an intermediate or end product by a microorganism. Metabolites can be small molecules of low molecular weight that influence biological processes and impact a variety of functions including, but not limited to, inhibitory effects on pathogens, catalytic activity, defensive mechanisms or other interactions with other organisms.

The metabolites with activity against fungal phytopathogens are, in the present context, termed “bioactive metabolites”. One group of bioactive metabolites are lipopeptides, such as iturins, fengycins and surfactins. Other groups of bioactive metabolites include, but are not limited to, polyketides and volatile compounds (VOCs).

### About

Wherever the term “about” is employed herein in the context of amounts, for example absolute amounts, such as numbers, purities, weights, concentrations, sizes, etc., or relative amounts (e.g. percentages, equivalents or ratios), timeframes, and parameters such as temperatures, pressure, etc., it will be appreciated that such variables are approximate and as such may vary by ±10%, for example ±5% and preferably ±2% (e.g. ±1%) from the actual numbers specified. This is the case even if such numbers are presented as percentages in the first place (for example ‘about 10%’ may mean±10% about the number 10, which is anything between 9% and 11%).

### Sequence Identity

In the present context, the term “sequence identity” is here defined as the sequence identity between proteins at the amino acid level. The protein sequence identity may be determined by comparing the amino acid sequence in a given position in each sequence when the sequences are aligned.

To determine the percent identity of two amino acid sequences, the sequences are aligned for optimal comparison purposes (e.g. gaps may be introduced in the sequence of a first amino acid sequence for optimal alignment with a second amino acid sequence). The amino acid residues at corresponding amino acid positions are then compared. When a position in the first sequence is occupied by the same amino acid residue as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100).

In one embodiment, the two sequences are the same length. In another embodiment, the two sequences are of different length and gaps are seen as different positions.

One may manually align the sequences and count the number of identical amino acids. Alternatively, alignment of two sequences for the determination of percent identity may be accomplished using a mathematical algorithm. Such an algorithm is incorporated into the XBLAST program of (Altschul et al. 1990). BLAST protein searches may be performed with the XBLAST program, score=50, wordlength=3 to obtain amino acid sequences homologous to a protein molecule of the invention.

To obtain gapped alignments for comparison purposes, Gapped BLAST may be utilized. Alternatively, PSI-Blast may be used to perform an iterated search, which detects distant relationships between molecules. When utilizing the XBLAST and Gapped BLAST programs, the default parameters of the respective programs may be used. See http://www.ncbi.nlm.nih.gov. Alternatively, sequence identity may be calculated after the sequences have been aligned e.g. by the BLAST program in the EMBL database (www.ncbi.nlm.gov/cgi-bin/BLAST). Generally, the default settings with respect to e.g. “scoring matrix” and “gap penalty” may be used for alignment.

The percent identity between two sequences may be determined using techniques similar to those described above, with or without allowing gaps. In calculating percent identity, only exact matches are counted.

### Derivative Strains of Bacillus

Plantations are globally faced with the challenge of maximizing agricultural yield in order to meet the steep global demands of large quantities of goods produced in an environmentally satisfactory manner. However, phytopathogens remains a significant issue causing devastating losses in yield worldwide. While phytopathogens may be combatted with some effect by application of chemicals such as fungicides, these solutions are not preferred as the applied compounds may have hazardous effects on human and animal health as well as negatively impacting the environment. Therefore, solutions based on application of natural organisms are in demand.

Biobased agriculture or food industries mainly rely on natural methods of classical strain improvement (CSI) techniques to create improved strains and products. This approach is guided by introduction of random mutations to a parental strain followed by screening and selection of improved variants. Mutations of classical strain techniques are random by nature and can be either natural or induced. Accordingly, the entire genome of the parental strain is probed in contrast to modern era site-directed genome engineering, such as CRISPR, affecting exclusively specific target genes. A benefit hereof is that improved complex phenotypes which may be governed by the interaction between multiple genes can be identified. In absence of thorough understanding of the parental strain genome, such types of improved complex phenotypes are unlikely to be identified by specific genomic substitutions. Moreover, strains developed by the classical strain improvement approach are considered to be non-genetically modified organisms (GMO) which negates the commercial barriers caused by the strict GMO regulations of for example the EU.

The derivative strains of Bacillus disclosed herein are obtained by induced mutagenesis of a parental strain with desired traits. Induced mutagenesis may be accomplished by direct exposure of the parental Bacillus strain to a mutagen to purposefully introduce mutations in the genome. The parental Bacillus strain utilized herein is a first-generation Bacillus amyloliquefaciens strain already adapted for inhibition of the fungal pathogen Fusarium graminearum and Botrytis cinerea.

Herein are disclosed second-generation derivatives obtained from that parental Bacillus strain which through mutagenesis have acquired increased inhibitory effect on phytopathogens, such as Fusarium graminearum and/or Botrytis cinerea. It is contemplated that the derivative strains of Bacillus amyloliquefaciens will aid in combatting the challenges of improving plant health and crop yield in an environmentally compelling manner.

Thus, an aspect of the present invention relates to a Bacillus amyloliquefaciens strain, or a mutant or variant thereof, with increased inhibitory effect on one or more phytopathogens compared to a parental strain of Bacillus amyloliquefaciens, wherein the Bacillus amyloliquefaciens strain, or a mutant or variant thereof, is a derivative strain of said parental strain of Bacillus amyloliquefaciens.

It is to be understood that derivative Bacillus amyloliquefaciens strains with identical or similar phenotypes also forms part of the invention. These may be obtained by the method described herein or by further evolution of derivative stains disclosed herein producing new mutants or variants with identical or similar phenotypes. Such strains may be said to have all of the identifying characteristics of the derivative strains disclosed herein. Accordingly, strains sharing all identifying characteristics can have different non-identical genomic sequences. The identifying characteristics may include, but is not limited to, the ability to inhibit a phytopathogen and/or increased production of one or more metabolites.

Thus, an embodiment of the present invention relates to a Bacillus amyloliquefaciens strain, or a mutant or variant thereof with all the identifying characteristics thereof, with increased inhibitory effect on one or more phytopathogens compared to a parental strain of Bacillus amyloliquefaciens.

Another embodiment of the present invention relates to the Bacillus amyloliquefaciens strain as described herein, wherein said Bacillus amyloliquefaciens strain has increased production of one or more metabolites compared to said parental strain of Bacillus amyloliquefaciens, when cultured under the same conditions.

Bacillus strains produce through a complex multifunctional enzyme system a range of bioactive metabolites. One group of bioactive metabolites are the lipopeptides, which consist of lipids connected to peptides. Lipopeptides acts as biosurfactants and may have antibiotic activity, e.g. fungicidal activity.

Therefore, an embodiment of the present invention relates to the Bacillus amyloliquefaciens strain as described herein, wherein said one or more metabolites are lipopeptides.

A group of lipopeptides known to have antibiotic activity are the cyclic lipopeptides. This group includes iturins, fengycins and surfactins, which all share a common structure consisting of a lipid tail linked to a short cyclic peptide. The variants of compounds in each group come from different amino acid components. Iturins and fengycins are known to have strong antifungal activity, whereas surfactins do not on their own exhibit great antifungal toxicity. However, surfactins may promote the antifungal activity of other lipopeptides.

Therefore, an embodiment of the present invention relates to the Bacillus amyloliquefaciens strain as described herein, wherein said lipopeptides are selected from the group consisting of iturins, fengycins and surfactins, and combinations thereof.

Another embodiment of the present invention relates to the Bacillus amyloliquefaciens strain as described herein, wherein said Bacillus amyloliquefaciens strain has increased production of iturins compared to said parental strain of Bacillus amyloliquefaciens.

A further embodiment of the present invention relates to the Bacillus amyloliquefaciens strain as described herein, wherein said Bacillus amyloliquefaciens strain has increased production of fengycins compared to said parental strain of Bacillus amyloliquefaciens.

A still further embodiment of the present invention relates to the Bacillus amyloliquefaciens strain as described herein, wherein said Bacillus amyloliquefaciens strain has increased production of surfactins compared to said parental strain of Bacillus amyloliquefaciens.

An even further embodiment of the present invention relates to the Bacillus amyloliquefaciens strain as described herein, wherein said Bacillus amyloliquefaciens strain has increased production of iturins and fengycins compared to said parental strain of Bacillus amyloliquefaciens.

Yet another embodiment of the present invention relates to the Bacillus amyloliquefaciens strain as described herein, wherein said Bacillus amyloliquefaciens strain has increased production of iturins, fengycins and surfactins compared to said parental strain of Bacillus amyloliquefaciens.

The development of improved Bacillus amyloliquefaciens strains with great inhibitory activity on phytopathogens is based on a parental strain which is subjected to induced mutagenesis. The parental strain thus constitutes the starting point for the evolution scheme and is selected based on favorable phenotypes, such as strains with a high baseline of phytopathogenic inhibitory activity. Thus, it is preferred to use parental strains with high antibiotic activity, such as high antifungal activity.

Therefore, an embodiment of the present invention relates to the Bacillus amyloliquefaciens strain as described herein, wherein said parental strain of Bacillus amyloliquefaciens (FHB-P) is deposited as DSM34003 at Deutsche Sammlung von Mikroorganismen und Zellkulturen GmbH (DSMZ), Inhoffenstr. 7B, D-38124 Braunschweig, Germany, by Chr. Hansen A/S, Hørsholm, Denmark on 24 Aug. 2021, wherein the Bacillus amyloliquefaciens strain, or a mutant or variant thereof, is a derivative strain of FHB-P.

The derivative strains of Bacillus amyloliquefaciens is developed by classical strain improvement. More specifically, the derivative strains disclosed herein are the product of an evolution campaign wherein mutations are randomly acquired in the parental strain followed by careful selection and analysis of candidate derivative strains. The random mutations can take the form of small-scale mutations, which typically affect a gene in one or a few nucleotides.

Thus, an embodiment of the present invention relates to the Bacillus amyloliquefaciens strain as described herein, wherein said Bacillus amyloliquefaciens strain is genetically distinct from said parental strain of Bacillus amyloliquefaciens.

Another embodiment of the present invention relates to the Bacillus amyloliquefaciens strain as described herein, wherein said Bacillus amyloliquefaciens strain comprises one or more mutations compared to said parental strain of Bacillus amyloliquefaciens.

A further embodiment of the present invention relates to the Bacillus amyloliquefaciens strain as described herein, wherein said one or more mutations are deletion(s), substitution(s) and/or insertion(s).

Especially bioactive metabolites produced by Bacillus strains contribute to their antibiotic capabilities. Derivative strains with alterations in the metabolite production or any relationship therewith is of interest.

Therefore, an embodiment of the present invention relates to the Bacillus amyloliquefaciens strain as described herein, wherein said one or more mutations affects the expression and/or the pathway of said one or more metabolites.

Without being bound by theory, it is contemplated that genetic changes in the proteasome components (e.g. ClpP or ClpC genes) contribute to high bioactive metabolite levels. These proteasome components play a direct role in the overall proteolysis of misfolded proteins in Bacillus subtilis and energy-dependent proteolysis in vivo is executed by the ClpCP and ClpXP proteases. Besides its global function in removal of damaged proteins, levels of central regulatory proteins are controlled by ClpPC. In Bacillus subtilis, the response regulator DegU and its cognate kinase, DegS, constitute a two-component system that regulates many cellular processes, including exoprotease production and genetic competence. It is therefore contemplated that DegU plays a critical role in regulating the expression of the biosynthetic pathways for bioactive metabolites, in particular compounds of the iturin and fengycin families. Phosphorylated DegU (DegU-P) activates its own promoter and is degraded by the ClpCP protease.

In addition, Bacillus subtilis Clp proteins are required for cell division and several stationary-phase phenomena, such as motility and degradative enzyme synthesis, as well as the development of sporulation and genetic competence.

In Bacillus subtilis the combined activity of a protein arginine kinase and phosphatase allows a rapid and reversible regulation of protein activity, and that protein arginine phosphorylation can play a physiologically important and regulatory role in bacteria. Interestingly, ClpCP activity is regulated through a McsB-dependent phosphorylation on two arginine residues and arginine phosphorylation plays a significant role for many other regulatory processes within the bacterial cell.

Among the genetic changes identified in the ClpPC primary sequences of the derivative strains disclosed herein, two arginine residues had been exchanged by different amino acids, which could result in a deregulation of their proteolytic activity. Moreover, derivative strains disclosed herein differed also from the parental strain by modification in the bacterial protein arginine kinase, McsB.

It is also contemplated that genetic changes in the alternative sigma factor SigH could be of importance as selected derivative strains with modification therein showed levels of metabolites and bioactivity above the parental strain. Single amino acid changes in SigH result in a not fully active version of this sigma factor. In contrast, alternative amino acid changes can suppress the poor interaction of SigH with the RNA polymerase, resulting in derivative strains with increased sporulation under high salinity stress conditions. Amino acid changes identified within the SigH sequence in the improved derivatives are located most likely within the regions 2.4 to 3.0 of the sigma factor. These regions are involved in promoter recognition and binding to the RNA polymerase, respectively. Therefore, those genetic changes might alter expression of genes regulated by this sigma factor.

By altering the expression and activation of Spo0A and ComA, genetic changes in SigH could results in an extension of the competence growth phase and a delayed entry into sporulation. This would result in an extended phase for bioactive metabolite production before cells enter sporulation. Moreover, the regulatory circuit involving ComA and Spo0A would also impact directly the activity of DegU and the expression of the surfactin pathway.

Indeed, some of the derivative strains disclosed herein with increased lipopeptide production comprised modifications in the SigH gene.

It is further contemplated that genetic changes in the aspartate phosphatases, rapI and rapC could be related to the improved production of lipopeptides of selected derivative strains. Both RapI and RapC constitute response regulators of two-component systems that regulate the activity of Spo0A and ComA, global regulators of multicellular behaviours such as entry into sporulation and competence development in Bacilli. RapI regulates the activity of the phosphotransferase Spo0F, which forms part of the phosphorelay system that controls Spo0A phosphorylation level. RapC acts antagonistically to the ComQXPA quorum sensing system as phosphatase or anti-activator of ComA~P, regulating competence development. Once again, selected genetic changes may impact the extension of the competence growth phase and delay entry into sporulation, which could explain the increased bioactive metabolite production in the improved derivatives.

It is also contemplated that modifications in the ATP-dependent zinc metallopeptidase FtsH contribute to high bioactive metabolite levels. FtsH is an ATP-dependent zinc metallopeptidase for both cytoplasmic and membrane proteins. It plays a role in the quality control of integral membrane proteins. The ftsH gene of Bacillus subtilis is involved in major cellular processes such as sporulation, stress adaptation and secretion.

Thus, an embodiment of the present invention relates to the Bacillus amyloliquefaciens strain as described herein, wherein said one or more mutations are located in one or more genes of said parental strain of Bacillus amyloliquefaciens selected from the group consisting of clpP, swrC, clpC, sigH, graR, ftsH, pdhC, degU, mcsB, rapI, and rapC, and combinations thereof.

Another embodiment of the present invention relates to the Bacillus amyloliquefaciens strain as described herein, wherein said one or more mutations are located in one or more genes of said parental strain of Bacillus amyloliquefaciens comprising a nucleic acid sequence selected from the group consisting of SEQ ID NO:1 (clpP), SEQ ID NO:2 (swrC), SEQ ID NO:3 (clpC), SEQ ID NO:4 (sigH), SEQ ID NO:5 (graR), SEQ ID NO:6 (ftsH), SEQ ID NO:7 (pdhC), SEQ ID NO:8 (degU), SEQ ID NO:9 (mcsB), SEQ ID NO:10 (rapI), and SEQ ID NO:11 (rapC) and combinations thereof.

Yet another embodiment of the present invention relates to the Bacillus amyloliquefaciens strain as described herein, wherein said one or more mutations are located in one or more genes of said parental strain of Bacillus amyloliquefaciens selected from the group consisting of clpP, clpC, sigH, and ftsH, and combinations thereof.

A further embodiment of the present invention relates to the Bacillus amyloliquefaciens strain as described herein, wherein said one or more mutations comprises at least one mutation in rapI and/or rapC.

A still further embodiment of the present invention relates to the Bacillus amyloliquefaciens strain as described herein, wherein said one or more mutations lead to one or more modifications in one or more proteins of said parental strain of Bacillus amyloliquefaciens comprising an amino acid sequence selected from the group consisting of SEQ ID NO:12 (ClpP), SEQ ID NO:13 (SwrC), SEQ ID NO:14 (ClpC), SEQ ID NO:15 (SigH), SEQ ID NO:16 (GraR), SEQ ID NO:17 (FtsH), SEQ ID NO:18 (PdhC), SEQ ID NO:19 (DegU), SEQ ID NO:20 (McsB), SEQ ID NO:21 (RapI), and SEQ ID NO:22 (RapC), and combinations thereof.

Some mutations of the derivative strains disclosed herein has been identified as favorable for improved metabolite production and/or inhibitory capacity on fungi. These mutations may be stand-alone mutations or be combined to yield improved derivative strains with favorable traits.

Thus, an embodiment of the present invention relates to the Bacillus amyloliquefaciens strain as described herein, wherein said one or more mutations lead to one or more modifications compared to said parental strain of Bacillus amyloliquefaciens selected from the group consisting of ClpP-R23H, ClpP-L1151, ClpC-R261C, ClpC-D380N, SigH-H28Y, SigH-A90T, SigH-A90V, SigH-H110Y, SigH-V461, GraR-E47K, PdhC-A106T, PdhC-E102K, DegU-A164V, McsB-A142V, FtsH-V80I, RapI-A241T and RapC-G190D, and combinations thereof.

Another embodiment of the present invention relates to the Bacillus amyloliquefaciens strain as described herein, wherein said one or more mutations are located in the clpP and/or swrC gene(s) of said parental strain of Bacillus amyloliquefaciens.

A further embodiment of the present invention relates to the Bacillus amyloliquefaciens strain as described herein, wherein said one or more mutations lead to one or more modifications compared to said parental strain of Bacillus amyloliquefaciens selected from the group consisting of ClpP-R23H, ClpP-L1151, and SwrC-L197F, and combinations thereof.

A still further embodiment of the present invention relates to the Bacillus amyloliquefaciens strain as described herein, wherein said one or more mutations lead to one or more modifications compared to said parental strain of Bacillus amyloliquefaciens selected from RapI-A241T and/or RapC-G190D.

The method for developing the derivative strains is adapted so that the number of acquired mutations are kept so low as so to produce a small number of single nucleotide polymorphisms (SNPs) in the parental strain rather than more radical derivative strains with high mutation rates.

Thus, an embodiment of the present invention relates to the Bacillus amyloliquefaciens strain as described herein, wherein the genome of said Bacillus amyloliquefaciens strain is at least 95%, such as at least 98%, such as at least 99%, such as at least 99.5%, such as at least 99.8%, such as at least 99.9% identical to the genome of said parental strain of Bacillus amyloliquefaciens.

Some particularly improved derivative strains have been identified as part of the evolution campaign, wherein mutagenesis is induced upon the parental strain and careful selection and analysis are performed on isolated derivative strains.

Therefore, an embodiment of the present invention relates to the Bacillus amyloliquefaciens strain as described herein, wherein said Bacillus amyloliquefaciens strain (FHB-S31) is deposited as DSM34007 at Deutsche Sammlung von Mikroorganismen und Zellkulturen GmbH (DSMZ), Inhoffenstr. 7B, D-38124 Braunschweig, Germany, by Chr. Hansen A/S, Hørsholm, Denmark on 24 Aug. 2021.

Another embodiment of the present invention relates to the Bacillus amyloliquefaciens strain as described herein, wherein said Bacillus amyloliquefaciens strain (FHB-S4) is deposited as DSM34006 at Deutsche Sammlung von Mikroorganismen und Zellkulturen GmbH (DSMZ), Inhoffenstr. 7B, D-38124 Braunschweig, Germany, by Chr. Hansen A/S, Hørsholm, Denmark on 24 Aug. 2021.

A further embodiment of the present invention relates to the Bacillus amyloliquefaciens strain as described herein, wherein said Bacillus amyloliquefaciens strain (FHB-S21) is deposited as DSM34318 at Deutsche Sammlung von Mikroorganismen und Zellkulturen GmbH (DSMZ), Inhoffenstr. 7B, D-38124 Braunschweig, Germany, by Chr. Hansen A/S, Hørsholm, Denmark on 30 Jun. 2022.

A still further embodiment of the present invention relates to the Bacillus amyloliquefaciens strain as described herein, wherein said Bacillus amyloliquefaciens strain (FHB-S11) is deposited as DSM34317 at Deutsche Sammlung von Mikroorganismen und Zellkulturen GmbH (DSMZ), Inhoffenstr. 7B, D-38124 Braunschweig, Germany, by Chr. Hansen A/S, Hørsholm, Denmark on 30 Jun. 2022.

The derivative strains disclosed herein have high inhibitory activity against phytopathogens that are known to commonly cause disease to plants and reduce health and yield of crops. These phytopathogens include a range of plant fungal and plant bacterial pathogens, and in particular those belonging to the genus Fusarium and/or Botrytis. Without being bound by theory, it is contemplated that primarily the high production of bioactive metabolites, such as lipopeptides, are responsible for the increased antibiotic activity of the derivative strains of Bacillus amyloliquefaciens

Accordingly, an embodiment of the present invention relates to the Bacillus amyloliquefaciens strain as described herein, wherein said phytopathogen is a plant fungal pathogen or a plant bacterial pathogen.

Another embodiment of the present invention relates to the Bacillus amyloliquefaciens strain as described herein, wherein said one or more phytopathogen(s) is from a genus selected from the group consisting of Fusarium, Botrytis, Erwinia, Dickeya, Agrobacterium, Xanthomonas, Xylella, Candidatus, Sclerotinia, Cercospora/Cercosporidium, Uncinula, Podosphaera, Phomopsis, Alternaria, Pseudomonas, Phytophthora, Phakopsora, Aspergillus, Uromyces, Cladosporium, Rhizopus, Penicillium, Rhizoctonia, Macrophomina, Mycosphaerella, Magnaporthe, Monilinia, Colletotrichum, Diaporthe, Corynespora, Gymnosporangium, Schizothyrium, Gloeodes, Botryosphaeria, Neofabraea, Wilsonomyces, Sphaerotheca, Erysiphe, Stagonospora, Pythium, Venturia, Verticillium, Ustilago, Claviceps, Tilletia, Phoma, Cochliobolus, Gaeumanomyces, Rhychosporium, Biopolaris, and Helminthosporium, and combinations thereof.

A further embodiment of the present invention relates to the Bacillus amyloliquefaciens strain as described herein, wherein said one or more phytopathogen(s) is a species selected from the group consisting of Botrytis cinerea, Botrytis squamosa, Erwinia carotovora, Erwinia amylovora, Dickeya dadantii, Dickeya solani, Agrobacterium tumefaciens, Xanthomonas axonopodis, Xanthomonas campestris pv. carotae, Xanthomonas pruni, Xanthomonas arboricola, Xanthomonas oryzae pv. oryzae, Xylella fastidiosa, Candidatus liberibacter, Fusarium culmorum, Fusarium graminearum, Fusarium oxysporum, Fusarium oxysporum f. sp. Cubense, Fusarium oxysporum f. sp. Lycopersici, Fusarium virguliforme, Sclerotinia sclerotiorum, Sclerotinia minor, Sclerotinia homeocarpa, Uncinula necator, Podosphaera leucotricha, Podosphaera clandestine, Phomopsis viticola, Alternaria tenuissima, Alternaria porri, Alternaria alternate, Alternaria solani, Alternaria tenuis, Pseudomonas syringae pv. Tomato, Phytophthora infestans, Phytophthora parasitica, Phytophthora sojae, Phytophthora capsici, Phytophthora cinnamon, Phytophthora fragariae, Phytophthora ramorum, Phytophthora palmivara, Phytophthora nicotianae, Phakopsora pachyrhizi, Phakopsora meibomiae, Aspergillus flavus, Aspergillus niger, Uromyces appendiculatus, Cladosporium herbarum, Rhizopus arrhizus, Rhizoctonia solani, Rhizoctonia zeae, Rhizoctonia oryzae, Rhizoctonia caritae, Rhizoctonia cerealis, Rhizoctonia crocorum, Rhizoctonia fragariae, Rhizoctonia ramicola, Rhizoctonia rubi, Rhizoctonia leguminicola, Macrophomina phaseolina, Mycosphaerella graminocola, Mycosphaerella fijiensis, Mycosphaerella pomi, Mycosphaerella citri, Magnaporthe oryzae, Magnaporthe grisea, Monilinia fruticola, Monilinia vacciniicorymbosi, Monilinia laxa, Colletotrichum gloeosporiodes, Colletotrichum acutatum, Coletotrichum candidum, Diaporthe citri, Corynespora cassiicola, Gymnosporangium juniperi-virginianae, Schizothyrium pomi, Gloeodes pomigena, Botryosphaeria dothidea, Wilsonomyces carpophilus, Sphaerotheca macularis, Sphaerotheca pannosa, Stagonospora nodorum, Pythium ultimum, Pythium aphanidermatum, Pythium irregularum, Pythium ulosum, Pythium lutriarium, Pythium sylvatium, Venturia inaequalis, Ustilago nuda, Ustilago maydis, Ustilago scitaminea, Claviceps pupurea, Tilletia tritici, Tilletia laevis, Tilletia horrid, Tilletia controversa, Phoma glycinicola, Phoma exigua, Phoma lingam, Cochliobolus sativus, Gaeumanomyces gaminis, Rhychosporium secalis, Helminthosporium secalis, Helminthosporium maydis, Helminthosporium solani, and Helminthosporium triticirepentis, and combinations thereof.

A still further embodiment of the present invention relates to the Bacillus amyloliquefaciens strain as described herein, wherein said one or more phytopathogen(s) is Fusarium graminearum, Fusarium culmorum and/or Botrytis cinerea.

A still further embodiment of the present invention relates to the Bacillus amyloliquefaciens strain as described herein, wherein said phytopathogen is Fusarium graminearum or Fusarium culmorum.

An even further embodiment of the present invention relates to the Bacillus amyloliquefaciens strain as described herein, wherein said phytopathogen is Fusarium graminearum.

The derivative strains may also have biofungicidal activity against fungi of the genus Botrytis. Accordingly, the derivative strains may be used to combat more than a single phytopathogen.

Therefore, an embodiment of the present invention relates to the Bacillus amyloliquefaciens strain as described herein, wherein said phytopathogen is Botrytis cinerea

Another embodiment of the present invention relates to the Bacillus amyloliquefaciens strain as described herein, wherein said one or more phytopathogens are Fusarium graminearum and Botrytis cinerea.

Gram-positive bacteria, such as Bacillus, are capable of forming spores, typically in the form of intracellular spores called endospores, as a surviving mechanism. These endospores are very retractile and thick-walled structures that constitute the most dormant form of bacteria as they exhibit minimal metabolism, respiration and enzyme production. Such bacterial spores are highly resistant to temperature fluctuations, chemical agents, UV radiation, pH gradients, drought and nutrition depletion. As the surrounding environment favors bacterial proliferation, the bacterial spores will germinate back into vegetative cells, i.e. an active bacterial cell undergoing metabolism. Accordingly, spore-forming bacteria are preferred in the present context as they possess the ability to lay dormant if conditions in the field does not favor survival. Thus, this risk of losing the biostimulant Bacillus amyloliquefaciens strain after application to the plant is reduced for spore-forming bacteria. Accordingly, the derivative strains disclosed herein have been positively selected for spore-formation ability.

Therefore, an embodiment of the present invention relates to the Bacillus amyloliquefaciens strain as described herein, wherein the Bacillus amyloliquefaciens strain is in the form of spores or vegetative cells, preferably spores.

The derivative strains of Bacillus amyloliquefaciens disclosed herein have been developed by classical strain improvement (CSI), and in particular by induced mutagenesis facilitated by exposure to a mutagen. An advantage of this type of random mutagenesis evolution is that a large spectrum of genes and the interrelations are probed as opposed to site-directed approaches wherein only few, but specific genes are mutated. In this manner it is possible to identify even complex phenotypes based and the CSI approach is besides plant disease combating used to develop strains adapted to certain abiotic conditions or capable of promoting growth of crops. The CSI approach includes the steps of providing a parental strain as a starting point, creating a library of derivative strains of high genetic diversity, and screening for desired target properties. The screening typically involves some assay to assert the target properties followed by the selection of lead candidate derivative strains.

Therefore, an aspect of the present invention relates to a method for preparing a Bacillus amyloliquefaciens strain according to any one of the preceding items, said method comprising the steps of:
(i) providing a parental strain of Bacillus amyloliquefaciens;
(ii) subjecting said parental strain of Bacillus amyloliquefaciens to one or more mutagens to generate one or more Bacillus amyloliquefaciens strains;
(iii) determine the inhibitory effect and/or production of one or more metabolites of said one or more Bacillus amyloliquefaciens strains; and
(iv) selecting a Bacillus amyloliquefaciens strain with increased inhibitory effect and/or increased production of one or more metabolites.

As described herein, the Bacillus amyloliquefaciens strains identified by the method disclosed herein have increased inhibitory effect on a range of phytopathogens, including plant fungal pathogen or a plant bacterial pathogen. Particularly, the Bacillus amyloliquefaciens strains are effective in inhibiting pathogens of the Fusarium genus, such as Fusarium graminearum and Fusarium culmorum, and/or of the Botrytis genus, such as Botrytis cinerea.

Thus, an embodiment of the present invention relates to the method as described herein, wherein said phytopathogen is Fusarium graminearum.

Another embodiment of the present invention relates to the method as described herein, wherein said phytopathogen is Botrytis cinerea.

To improve the success rate of improved identifying derivative strains by CSI, a strategy is to use a parental strain with a phenotype that is already enhanced in a set of target properties. In the present context it may therefore be favorable to select a parental strain of Bacillus amyloliquefaciens with high inhibitory activity against phytopathogens. Thus, an embodiment of the present invention relates to the method as described herein, wherein said parental strain of Bacillus amyloliquefaciens has increased production of one or more metabolites, such as lipopeptides (e.g. iturins, fengycins and surfactin) compared to a wild type Bacillus amyloliquefaciens strain.

Another embodiment of the present invention relates to the method as described herein, wherein said parental strain of Bacillus amyloliquefaciens (FHB-P) is deposited as DSM34003 at Deutsche Sammlung von Mikroorganismen und Zellkulturen GmbH (DSMZ), Inhoffenstr. 7B, D-38124 Braunschweig, Germany, by Chr. Hansen A/S, Hørsholm, Denmark on 24 Aug. 2021.

The parental strain is exposed to a mutagen to facilitate the preparation of a diverse library of derivative strains through induced mutagenesis. The mutagen may be either a physical or chemical mutagen, but common for both is that the degree of exposure may be adjusted to achieve a desired number of acquired mutations. To obtain the desired mutational rate, the type of mutagen as well as exposure time and/or concentration or intensity can be optimized for the specific parental strain.

Accordingly, an embodiment of the present invention relates to the method as described herein, wherein said one or more mutagens are chemical mutagens and/or physical mutagens.

Another embodiment of the present invention relates to the method as described herein, wherein said one or more chemical mutagens are selected from ethyl methane sulphonate (EMS), alkylating agents (such as ethyl ethane sulphonate, methyl methane sulphonate, ethylene imines, etc.), base analogs, azides, and N-methyl-N′-nitro-N-nitroguanidine (MNNG), preferably EMS.

The appropriate concentration of mutagen can be balanced with the exposure time to the mutagen to achieve a killing rate percentage suitable for constructing a derivative strain library with a desired degree of mutation. The optimal conditions for the evolution campaign may vary vastly between different microorganism as the tolerance to the mutagen will differ not only between genus, but in different strains within a specific species.

Therefore, an embodiment of the present invention relates to the method as described herein, wherein the concentration of chemical mutagen is in the range of about 12-24 g/L mM, preferably in the range of about 18-24 g/L.

Another embodiment of the present invention relates to the method as described herein, wherein said parental strain of Bacillus amyloliquefaciens is subjected to said mutagen for a period of time in the range of about 60 min to about 180 min, such as 90 min to about 150 min, such as about 100 min to about 140 min.

A further embodiment of the present invention relates to the method as described herein, wherein said parental strain of Bacillus amyloliquefaciens is subjected to said mutagen for approximately 120 min.

Yet another embodiment of the present invention relates to the method as described herein, wherein said one or more physical mutagens are selected from UV-light, gamma-rays and X-rays.

The derivative strains can be screened and selected based on a set of target properties. Herein, the main attribute of the derivative strains is their inhibitory effect on phytopathogens. Thus, a relevant assay as part of the screening for lead candidate derivative strains is a growth inhibition assay. Such as growth inhibition assay may be performed by measuring the amount of phytopathogen over time by optical density (OD) in a test medium. Residual noise from the Bacillus amyloliquefaciens may be eliminated from the measurement by adding an antibiotic directed against Bacillus amyloliquefaciens to the sample.

Accordingly, an embodiment of the present invention relates to the method as described herein, wherein said inhibitory effect is determined by exposing said phytopathogen to a sample comprising a Bacillus amyloliquefaciens strain and measuring growth of said phytopathogen.

Another embodiment of the present invention relates to the method as described herein, wherein said sample further comprises one or more antibiotics.

A further embodiment of the present invention relates to the method as described herein, wherein said growth is determined by measuring the optical density (OD) of said phytopathogen.

Another attribute of the derivative strains that appears to be important for the antibiotic activity is the production of metabolites, including bioactive metabolites such as lipopeptides. Therefore, determination of the level of metabolite production may also be used to identify potential lead candidate derivative strains of Bacillus amyloliquefaciens.

Thus, an embodiment of the present invention relates to the method as described herein, wherein said one or more metabolites are lipopeptides.

A further embodiment of the present invention relates to the method as described herein, wherein said lipopeptides are selected from the group consisting of iturins, fengycins and surfactins, and combinations thereof.

A still further embodiment of the present invention relates to the method as described herein, wherein said production of one or more metabolites is quantified by liquid chromatography-mass spectrometry (LC-MS).

The method may involve an additional step of sequencing selected derivative strain to gain additional genetic knowledge and direct further development of derivative strains with improved inhibitory activity. Such genetic information may improve the understanding of the target genes and their interplay responsible for governing any target properties.

Therefore, an embodiment of the present invention relates to the method as described herein, wherein said method further comprises a step (v) of sequencing and identifying single nucleotide polymorphisms (SNPs) of said one or more Bacillus amyloliquefaciens strains selected in step (iv).

By executing the method disclosed herein it is possible to obtain derivative strains of Bacillus amyloliquefaciens with improved inhibitory activity against phytopathogens.

Thus, an aspect of the present invention relates to a Bacillus amyloliquefaciens strain obtainable by the method as described herein.

For most practical purposes the Bacillus amyloliquefaciens strain will be part of a composition comprising other components to improve stability, deliverability or otherwise improve the performance as a plant growth promoting agent. Many of such additional components may be standard ingredient that are typically used in formulations of plant growth promoting agents, plant biostimulants, or biofungicides.

Thus, an aspect of the present invention relates to a composition comprising:
(i) a Bacillus amyloliquefaciens strain as described herein, and/or
(ii) a fermentation product produced by a Bacillus amyloliquefaciens strain as described herein.

An embodiment of the present invention relates to the composition as described herein, wherein said composition further comprises one or more agrochemically acceptable excipients, carriers, surfactants, dispersants and yeast extracts.

Since secreted bioactive metabolites, such as lipopeptides, contribute to the inhibitory activity, the composition may include the fermentation product of said Bacillus amyloliquefaciens strain. Preferably, the composition comprises both the Bacillus amyloliquefaciens strain and the fermentation product thereof.

Thus, an embodiment of the present invention relates to the composition as described herein, wherein said composition comprises the fermentation product produced by the Bacillus amyloliquefaciens strain.

Another embodiment of the present invention relates to the composition as described herein, wherein said composition comprises the Bacillus amyloliquefaciens strain.

A preferred embodiment of the present invention relates to the composition as described herein, wherein said composition comprises:
(i) the Bacillus amyloliquefaciens strain, and
(ii) the fermentation product produced by the Bacillus amyloliquefaciens strain.

The composition may preferably comprise said Bacillus amyloliquefaciens strain in the form of spores is this increases stability and longevity of the composition, especially when applied under harsh conditions, such as drought or the like.

Therefore, an embodiment of the present invention relates to the composition as described herein, wherein said composition comprises spores of said Bacillus amyloliquefaciens strain.

Besides any agrochemically acceptable standard ingredients, the composition may comprise additional active ingredients. Additional active ingredients may be, but is not necessarily, of different origin than microbial. They can increase the potency of the composition either by supplementing the inhibitory activity of the Bacillus amyloliquefaciens strain with a different effect or by working in synergy with the Bacillus amyloliquefaciens strain. Different effects include, but are not limited to, inhibition of other phytopathogens, such as insects or nematodes, or stimulating growth by provision of nutrients.

Thus, an embodiment of the present invention relates to the composition as described herein, wherein said composition further comprises one or more active ingredients.

Another embodiment of the present invention relates to the composition as described herein, wherein said one or more active ingredients are of microbial, biological or chemical origin.

A further embodiment of the present invention relates to the composition as described herein, wherein said one or more active ingredients are selected from the group consisting of an insecticide, fungicide, nematicide, bactericide, herbicide, plant extract, plant growth regulator, a plant growth stimulator, and fertilizer.

A still further embodiment of the present invention relates to the composition as described herein, wherein said insecticide is selected from the group consisting of pyrethroids, bifenthrin, tefluthrin, zeta-cypermethrin, organophosphates, chlorethoxyphos, chlorpyrifos, tebupirimphos, cyfluthrin, fiproles, fipronil, nicotinoids, and clothianidin, and combinations thereof.

An even further embodiment of the present invention relates to the composition as described herein, wherein said fungicide is selected from the group consisting of fluopyram plus tebuconazole, chlorothalonil, thiophanate-methyl, prothioconazole, metalaxyl, and copper hydroxide, and combinations thereof.

A preferred variant of the composition combines a derivative strain of Bacillus amyloliquefaciens as disclosed herein with a different strain of bacteria. The second bacterial strain may function as a plant biostimulant or plant growth promoting agent.

Thus, an embodiment of the present invention relates to the composition as described herein, wherein said one or more active ingredients are selected from another strain of bacteria different from said Bacillus amyloliquefaciens strain.

The composition disclosed herein may be in the form of a liquid, a wettable powder, a granule, a spreadable granule, a wettable granule, a microencapsulation, and a planting matrix or any technically feasible formulation that may include suitable agrochemically acceptable components. Alternatively, the composition may also be provided as an oil formulation, such as a water in oil (W/O) emulsion, an oil in water (O/W) emulsion, a microemulsion, or an oil dispersion. For foliar application of the composition to a plant it is preferred that the composition is in liquid form.

Thus, an embodiment of the present invention relates to the composition as described herein, wherein said composition is a form selected from the group consisting of a liquid, a wettable powder, a granule, a spreadable granule, a wettable granule, a microencapsulation, and a planting matrix.

Another embodiment of the present invention relates to the composition as described herein, wherein said composition is a liquid formulation.

To further improve the stability and longevity of the Bacillus amyloliquefaciens strain and any other sensitive ingredient or components included in the composition it may benefit to include a coating polymer. Such polymer may shield bacterial strains from hostile environment conditions. In one variant, bacterial spores of the Bacillus amyloliquefaciens strain is coated to improve durability of the microorganism.

Therefore, an embodiment of the present invention relates to the composition as described herein, wherein said composition further comprises a coating polymer.

The compositions may also comprise combinations of the derivative strains described herein to combat multiple phytopathogens simultaneously. In particular, it is contemplated that the compositions may comprise derivative strains with improved biofungicidal activity against fungi of the genus Fusarium and Botrytis, respectively. Thus, by example, a composition may comprise two derivative strains; a first derivative strains with high biofungicidal activity against Fusarium graminearum and a second derivative strains with high biofungicidal activity against Botrytis cinerea.

Accordingly, an embodiment of the present invention relates to the composition as described herein, wherein said compositions comprises two or more Bacillus amyloliquefaciens strains as described herein.

Another embodiment of the present invention relates to the composition as described herein, wherein said compositions comprises two Bacillus amyloliquefaciens strains as described herein.

The derivative strains of Bacillus amyloliquefaciens and compositions comprising them disclosed herein are suitable for application to plants with the aim of inhibiting the growth of phytopathogens and thereby limiting plants disease and improving plant health. The derivative strains and compositions are contemplated to be especially effective against fungi of the Fusarium genus.

Therefore, an aspect of the present invention relates to a method of inhibiting growth of one or more phytopathogens on a plant, said method comprising applying a Bacillus amyloliquefaciens strain or a composition as described herein to the plant or a part of the plant.

An embodiment of the present invention relates to the method as described herein, wherein said phytopathogen belongs to the Fusarium genus.

Another embodiment of the present invention relates to the method as described herein, wherein said phytopathogen is Fusarium graminearum or Fusarium culmorum.

A further embodiment of the present invention relates to the method as described herein, wherein said phytopathogen is Fusarium graminearum.

A still further embodiment of the present invention relates to the method as described herein, wherein said phytopathogen belongs to the Botrytis genus.

An even further embodiment of the present invention relates to the method as described herein, wherein said phytopathogen is Botrytis cinerea.

Plants that could be the beneficiary of the inhibitory effect of the derivative strains of Bacillus amyloliquefaciens on phytopathogens in principle include any plant that may attract a phytopathogen. Typically, the method of inhibiting growth of one or more phytopathogens on a plant as disclosed herein is mainly relevant for agriculture, because relatively small improvements in yield can make a great difference in an industrial setting. Moreover, the prospect of being able to improve yield in a climate-friendly manner is attractive and stands in stark contrast to the traditional image of modern agronomy polluting the environment with agrochemicals that cause widespread ecological damage.

Thus, an embodiment of the present invention relates to the method as described herein, wherein the plant is selected from the group consisting of a crop, a monocotyledonous plant, a dicotyledonous plant, a tree, an herb, a bush, a grass, a vine, a fern, a moss, and green algae.

Within this grouping of plants are found a large and diverse selection of plants that are commercialized in one way or another, which include, but is not limited to, corn, sweet corn, popcorn, seed corn, silage corn, field corn, rice, wheat, barley, sorghum, asparagus, berry, blueberry, blackberry, raspberry, loganberry, huckleberry, cranberry, gooseberry, elderberry, currant, caneberry, bush berry, brassica vegetables, broccoli, cabbage, cauliflower, brussels sprouts, collards, kale, mustard greens, kohlrabi, bulb vegetables, onion, garlic, shallots, citrus, orange, grapefruit, lemon, tangerine, tangelo, pomelo, fruiting vegetables, pepper, avocado, tomato, eggplant, ground cherry, tomatillo, okra, grape, herbs/spices, cucurbit vegetables, cucumber, cantaloupe, melon, muskmelon, squash, watermelon, pumpkin, leafy vegetables, lettuce, celery, spinach, parsley, radicchio, legumes/vegetables (succulent and dried beans and peas), beans, green beans, snap beans, shell beans, soybeans, dry beans, garbanzo beans, lima beans, peas, chick peas, split peas, lentils, oil seed crops, canola, castor, coconut, cotton, flax, oil palm, olive, peanut, rapeseed, safflower, sesame, sunflower, soybean, pome fruit, apple, crabapple, pear, quince, mayhaw, root/tuber and corm vegetables, carrot, potato, sweet potato, beets, ginger, horseradish, radish, ginseng, turnip, stone fruit, apricot, cherry, nectarine, peach, plum, prune, strawberry, tree nuts, almond, pistachio, pecan, walnut, filberts, chestnut, cashew, beechnut, butternut, macadamia, kiwi, banana, agave, tobacco, ornamental plants, poinsettia, hardwood cuttings, oak, maple, sugarcane, sugarbeet, grass, or turf grass. The method of inhibiting growth of one or more phytopathogens on a plant is applicable to any of these plants.

Another embodiment of the present invention relates to the method as described herein, wherein the plant is selected from the group consisting of wheat, barley, oats, small cereal grains, corn, rice, sugar cane, soybean, potato, carrot, coffee and banana.

The Bacillus amyloliquefaciens strain or composition is preferably applied directly to the plant or part of the plant to protect the plant against attacks by a phytopathogen. Application may be accomplished by spraying the Bacillus amyloliquefaciens strain or composition onto the foliage of the plants. Such application can typically be implemented in most agricultural settings without the need for investment in additional equipment.

Therefore, an embodiment of the present invention relates to the method as described herein, wherein the part of the plant is selected from the group consisting of a seed, fruit, root, stem, leaf, corm, tuber, bulb and rhizome.

Another embodiment of the present invention relates to the method as described herein, wherein said Bacillus amyloliquefaciens strain or composition is applied to the foliage of the plant.

A further embodiment of the present invention relates to the method as described herein, wherein said Bacillus amyloliquefaciens strain or composition is sprayed onto the plant.

The Bacillus amyloliquefaciens strain or composition is utilized with the aim of inhibiting growth of phytopathogens on plants and thereby attain a series of positive effects as a consequence of the reduction of plant disease.

Therefore, an aspect of the present invention relates to the use of a Bacillus amyloliquefaciens strain, or a composition as described herein for inhibiting growth of one or more phytopathogens on a plant.

An embodiment of the present invention relates to the use as described herein, wherein inhibition of Fusarium graminearum, Fusarium culmorum and/or Botrytis cinerea growth facilitates improved seedling vigor, improved root development, improved plant growth, improved plant health, increased yield, improved appearance, reduced pathogenic infection, or a combination thereof.

The Bacillus amyloliquefaciens strain or composition may conveniently be provided in a container together with any potential other active ingredients that are suitable for use together or sequentially with the Bacillus amyloliquefaciens strain or composition. Instructions may be included to guide the user in application and dosing of the Bacillus amyloliquefaciens strain or composition and any other active ingredient.

Accordingly, an aspect of the present invention relates to a kit comprising:
(i) a Bacillus amyloliquefaciens strain or a composition as described herein;
(ii) a container; and
(iii) optionally, instructions for use.

An embodiment of the present invention relates to the kit as described herein, wherein the kit further comprises one or more active ingredients selected from the group consisting of an insecticide, fungicide, nematicide, bactericide, herbicide, plant extract, plant growth regulator, a plant growth stimulator, and fertilizer.

Another embodiment of the present invention relates to the kit as described herein, wherein said Bacillus amyloliquefaciens strain and said one or more active ingredients are provided in separate compartments in the container.

The listing or discussion of an apparently prior published document in this specification should not necessarily be taken as an acknowledgement that the document is part of the state of the art or is common general knowledge.

Preferences, options and embodiments for a given aspect, feature or parameter of the invention should, unless the context indicates otherwise, be regarded as having been disclosed in combination with any and all preferences, options and embodiments for all other aspects, features and parameters of the invention. This is especially true for the description of the derivative strains and all its features, which may readily be part of the part of the method or use for inhibiting growth of a phytopathogen on a plant.

Embodiments and features of the present invention are also outlined in the following items.

### Items

Y1. A Bacillus amyloliquefaciens strain, or a mutant or variant thereof, with increased inhibitory effect on one or more phytopathogens compared to a parental strain of Bacillus amyloliquefaciens,
wherein the Bacillus amyloliquefaciens strain, or a mutant or variant thereof, is a derivative strain of said parental strain of Bacillus amyloliquefaciens.
Y2. The Bacillus amyloliquefaciens strain according to item Y1, wherein said Bacillus amyloliquefaciens strain has increased production of one or more metabolites compared to said parental strain of Bacillus amyloliquefaciens, when cultured under the same conditions.
Y3. The Bacillus amyloliquefaciens strain according to item Y2, wherein said one or more metabolites are lipopeptides.
Y4. The Bacillus amyloliquefaciens strain according to item Y3, wherein said lipopeptides are selected from the group consisting of iturins, fengycins and surfactins, and combinations thereof.
Y5. The Bacillus amyloliquefaciens strain according to any one of the preceding items, wherein said Bacillus amyloliquefaciens strain has increased production of iturins compared to said parental strain of Bacillus amyloliquefaciens.
Y6. The Bacillus amyloliquefaciens strain according to any one of the preceding items, wherein said Bacillus amyloliquefaciens strain has increased production of fengycins compared to said parental strain of Bacillus amyloliquefaciens.
Y7. The Bacillus amyloliquefaciens strain according to any one of the preceding items, wherein said Bacillus amyloliquefaciens strain has increased production of surfactins compared to said parental strain of Bacillus amyloliquefaciens.
Y8. The Bacillus amyloliquefaciens strain according to any one of the preceding items, wherein said Bacillus amyloliquefaciens strain has increased production of iturins and fengycins compared to said parental strain of Bacillus amyloliquefaciens.
Y9. The Bacillus amyloliquefaciens strain according to any one of the preceding items, wherein said Bacillus amyloliquefaciens strain has increased production of iturins, fengycins and surfactins compared to said parental strain of Bacillus amyloliquefaciens.
Y10. The Bacillus amyloliquefaciens strain according to any one of the preceding items, wherein said parental strain of Bacillus amyloliquefaciens (FHB-P) is deposited as DSM34003 at Deutsche Sammlung von Mikroorganismen und Zellkulturen GmbH (DSMZ), Inhoffenstr. 7B, D-38124 Braunschweig, Germany, by Chr. Hansen A/S, Hørsholm, Denmark on 24 Aug. 2021.
Y11. The Bacillus amyloliquefaciens strain according to any one of the preceding items, wherein said Bacillus amyloliquefaciens strain is genetically distinct from said parental strain of Bacillus amyloliquefaciens.
Y12. The Bacillus amyloliquefaciens strain according to any one of the preceding items, wherein said Bacillus amyloliquefaciens strain comprises one or more mutations compared to said parental strain of Bacillus amyloliquefaciens.
Y13. The Bacillus amyloliquefaciens strain according to item Y12, wherein said one or more mutations are deletion(s), substitution(s) and/or insertion(s).
Y14. The Bacillus amyloliquefaciens strain according to any one of items Y12 or Y13, wherein said one or more mutations affects the expression and/or the pathway of said one or more metabolites.
Y15. The Bacillus amyloliquefaciens strain according to any one of items Y12-Y14, wherein said one or more mutations are located in one or more genes of said parental strain of Bacillus amyloliquefaciens selected from the group consisting of clpP, swrC, clpC, sigH, graR, ftsH, pdhC, degU, mcsB, rapI, and rapC, and combinations thereof.
Y16. The Bacillus amyloliquefaciens strain according to any one of claims Y12-Y15, wherein said one or more mutations are located in one or more genes of said parental strain of Bacillus amyloliquefaciens comprising a nucleic acid sequence selected from the group consisting of SEQ ID NO:1 (clpP), SEQ ID NO:2 (swrC), SEQ ID NO:3 (clpC), SEQ ID NO:4 (sigH), SEQ ID NO:5 (graR), SEQ ID NO:6 (ftsH), SEQ ID NO:7 (pdhC), SEQ ID NO:8 (degU), SEQ ID NO:9 (mcsB), SEQ ID NO:10 (rapI), and SEQ ID NO:11 (rapC) and combinations thereof.
Y17. The Bacillus amyloliquefaciens strain according to any one of items Y12-Y16, wherein said one or more mutations are located in one or more genes of said parental strain of Bacillus amyloliquefaciens selected from the group consisting of clpP, clpC, sigH, and ftsH, and combinations thereof.
Y18. The Bacillus amyloliquefaciens strain according to any one of claims Y12-Y17, wherein said one or more mutations lead to one or more modifications in one or more proteins of said parental strain of Bacillus amyloliquefaciens comprising an amino acid sequence selected from the group consisting of SEQ ID NO:12 (ClpP), SEQ ID NO:13 (SwrC), SEQ ID NO:14 (ClpC), SEQ ID NO:15 (SigH), SEQ ID NO:16 (GraR), SEQ ID NO:17 (FtsH), SEQ ID NO:18 (PdhC), SEQ ID NO:19 (DegU), SEQ ID NO:20 (McsB), SEQ ID NO:21 (RapI), and SEQ ID NO:22 (RapC), and combinations thereof.
Y19. The Bacillus amyloliquefaciens strain according to any one of items Y12-Y18, wherein said one or more mutations lead to one or more modifications compared to said parental strain of Bacillus amyloliquefaciens selected from the group consisting of ClpP-R23H, ClpP-L1151, ClpC-R261C, ClpC-D380N, SigH-H28Y, SigH-A90T, SigH-A90V, SigH-H110Y, SigH-V461, GraR-E47K, PdhC-A106T, PdhC-E102K, DegU-A164V, McsB-A142V, FtsH-V80I, RapI-A241T and RapC-G190D, and combinations thereof.
Y20. The Bacillus amyloliquefaciens strain according to any one of items Y12-Y16, wherein said one or more mutations are located in the clpP and/or swrC gene(s) of said parental strain of Bacillus amyloliquefaciens.
Y21. The Bacillus amyloliquefaciens strain according to any one of items Y12-Y16 or 20, wherein said one or more mutations lead to one or more modifications compared to said parental strain of Bacillus amyloliquefaciens selected from the group consisting of ClpP-R23H, ClpP-L1151, and SwrC-L197F, and combinations thereof.
Y22. The Bacillus amyloliquefaciens strain according to any one of the preceding items, wherein said Bacillus amyloliquefaciens strain (FHB-S31) is deposited as DSM34007 at Deutsche Sammlung von Mikroorganismen und Zellkulturen GmbH (DSMZ), Inhoffenstr. 7B, D-38124 Braunschweig, Germany, by Chr. Hansen A/S, Hørsholm, Denmark on 24 Aug. 2021.
Y23. The Bacillus amyloliquefaciens strain according to any one of items Y1-Y21, wherein said Bacillus amyloliquefaciens strain (FHB-S4) is deposited as DSM34006 at Deutsche Sammlung von Mikroorganismen und Zellkulturen GmbH (DSMZ), Inhoffenstr. 7B, D-38124 Braunschweig, Germany, by Chr. Hansen A/S, Hørsholm, Denmark on 24 Aug. 2021.
Y24. The Bacillus amyloliquefaciens strain according to any one of items Y1-Y21, wherein said Bacillus amyloliquefaciens strain (FHB-S21) is deposited as DSM34318 at Deutsche Sammlung von Mikroorganismen und Zellkulturen GmbH (DSMZ), Inhoffenstr. 7B, D-38124 Braunschweig, Germany, by Chr. Hansen A/S, Hørsholm, Denmark on 30 Jun. 2022.
Y25. The Bacillus amyloliquefaciens strain according to any one of items Y1-Y21, wherein said Bacillus amyloliquefaciens strain (FHB-S11) is deposited as DSM34317 at Deutsche Sammlung von Mikroorganismen und Zellkulturen GmbH (DSMZ), Inhoffenstr. 7B, D-38124 Braunschweig, Germany, by Chr. Hansen A/S, Hørsholm, Denmark on 30 Jun. 2022.
Y26. The Bacillus amyloliquefaciens strain according to any one of the preceding items, wherein said phytopathogen is a plant fungal pathogen or a plant bacterial pathogen.
Y27. The Bacillus amyloliquefaciens strain according to any one of the preceding items, wherein said one or more phytopathogen(s) is from a genus selected from the group consisting of Fusarium, Botrytis, Erwinia, Dickeya, Agrobacterium, Xanthomonas, Xylella, Candidatus, Sclerotinia, Cercospora/Cercosporidium, Uncinula, Podosphaera, Phomopsis, Alternaria, Pseudomonas, Phytophthora, Phakopsora, Aspergillus, Uromyces, Cladosporium, Rhizopus, Penicillium, Rhizoctonia, Macrophomina, Mycosphaerella, Magnaporthe, Monilinia, Colletotrichum, Diaporthe, Corynespora, Gymnosporangium, Schizothyrium, Gloeodes, Botryosphaeria, Neofabraea, Wilsonomyces, Sphaerotheca, Erysiphe, Stagonospora, Pythium, Venturia, Verticillium, Ustilago, Claviceps, Tilletia, Phoma, Cochliobolus, Gaeumanomyces, Rhychosporium, Biopolaris, and Helminthosporium, and combinations thereof.
Y28. The Bacillus amyloliquefaciens strain according to any one of the preceding items, wherein said one or more phytopathogen(s) is a species selected from the group consisting of Botrytis cinerea, Botrytis squamosa, Erwinia carotovora, Erwinia amylovora, Dickeya dadantii, Dickeya solani, Agrobacterium tumefaciens, Xanthomonas axonopodis, Xanthomonas campestris pv. carotae, Xanthomonas pruni, Xanthomonas arboricola, Xanthomonas oryzae pv. oryzae, Xylella fastidiosa, Candidatus liberibacter, Fusarium culmorum, Fusarium graminearum, Fusarium oxysporum, Fusarium oxysporum f. sp. Cubense, Fusarium oxysporum f. sp. Lycopersici, Fusarium virguliforme, Sclerotinia sclerotiorum, Sclerotinia minor, Sclerotinia homeocarpa, Uncinula necator, Podosphaera leucotricha, Podosphaera clandestine, Phomopsis viticola, Alternaria tenuissima, Alternaria porri, Alternaria alternate, Alternaria solani, Alternaria tenuis, Pseudomonas syringae pv. Tomato, Phytophthora infestans, Phytophthora parasitica, Phytophthora sojae, Phytophthora capsici, Phytophthora cinnamon, Phytophthora fragariae, Phytophthora ramorum, Phytophthora palmivara, Phytophthora nicotianae, Phakopsora pachyrhizi, Phakopsora meibomiae, Aspergillus flavus, Aspergillus niger, Uromyces appendiculatus, Cladosporium herbarum, Rhizopus arrhizus, Rhizoctonia solani, Rhizoctonia zeae, Rhizoctonia oryzae, Rhizoctonia caritae, Rhizoctonia cerealis, Rhizoctonia crocorum, Rhizoctonia fragariae, Rhizoctonia ramicola, Rhizoctonia rubi, Rhizoctonia leguminicola, Macrophomina phaseolina, Mycosphaerella graminocola, Mycosphaerella fijiensis, Mycosphaerella pomi, Mycosphaerella citri, Magnaporthe oryzae, Magnaporthe grisea, Monilinia fruticola, Monilinia vacciniicorymbosi, Monilinia laxa, Colletotrichum gloeosporiodes, Colletotrichum acutatum, Coletotrichum candidum, Diaporthe citri, Corynespora cassiicola, Gymnosporangium juniperi-virginianae, Schizothyrium pomi, Gloeodes pomigena, Botryosphaeria dothidea, Wilsonomyces carpophilus, Sphaerotheca macularis, Sphaerotheca pannosa, Stagonospora nodorum, Pythium ultimum, Pythium aphanidermatum, Pythium irregularum, Pythium ulosum, Pythium lutriarium, Pythium sylvatium, Venturia inaequalis, Ustilago nuda, Ustilago maydis, Ustilago scitaminea, Claviceps pupurea, Tilletia tritici, Tilletia laevis, Tilletia horrid, Tilletia controversa, Phoma glycinicola, Phoma exigua, Phoma lingam, Cochliobolus sativus, Gaeumanomyces gaminis, Rhychosporium secalis, Helminthosporium secalis, Helminthosporium maydis, Helminthosporium solani, and Helminthosporium triticirepentis, and combinations thereof.
Y29. The Bacillus amyloliquefaciens strain according to any one of the preceding items, wherein said one or more phytopathogen(s) is Fusarium graminearum, Fusarium culmorum and/or Botrytis cinerea.
Y30. The Bacillus amyloliquefaciens strain according to any one of the preceding items, wherein said phytopathogen is Fusarium graminearum or Fusarium culmorum.
Y31. The Bacillus amyloliquefaciens strain according to any one of the preceding items, wherein said phytopathogen is Fusarium graminearum.
Y32. The Bacillus amyloliquefaciens strain according to items Y1-Y29, wherein said phytopathogen is Botrytis cinerea Y33. the Bacillus amyloliquefaciens strain according to items Y1-Y29, wherein said one or more phytopathogens are Fusarium graminearum and Botrytis cinerea.
Y34. The Bacillus amyloliquefaciens strain according to any one of the preceding items, wherein the Bacillus amyloliquefaciens strain is in the form of spores or vegetative cells, preferably spores.
X1. A method for preparing a Bacillus amyloliquefaciens strain according to any one of the preceding items, said method comprising the steps of:
   (i) providing a parental strain of Bacillus amyloliquefaciens;
   (ii) subjecting said parental strain of Bacillus amyloliquefaciens to one or more mutagens to generate one or more Bacillus amyloliquefaciens strains;
   (iii) determine the inhibitory effect and/or production of one or more metabolites of said one or more Bacillus amyloliquefaciens strains; and
   (iv) selecting a Bacillus amyloliquefaciens strain with increased inhibitory effect and/or increased production of one or more metabolites.
X2. The method according to item X1, wherein the one or more phytopathogens are selected from a plant fungal pathogen or a plant bacterial pathogen.
X3. The method according to any one of items X1 or X2, wherein said one or more phytopathogen(s) is from a genus selected from the group consisting of Fusarium, Botrytis, Erwinia, Dickeya, Agrobacterium, Xanthomonas, Xylella, Candidatus, Sclerotinia, Cercospora/Cercosporidium, Uncinula, Podosphaera, Phomopsis, Alternaria, Pseudomonas, Phytophthora, Phakopsora, Aspergillus, Uromyces, Cladosporium, Rhizopus, Penicillium, Rhizoctonia, Macrophomina, Mycosphaerella, Magnaporthe, Monilinia, Colletotrichum, Diaporthe, Corynespora, Gymnosporangium, Schizothyrium, Gloeodes, Botryosphaeria, Neofabraea, Wilsonomyces, Sphaerotheca, Erysiphe, Stagonospora, Pythium, Venturia, Verticillium, Ustilago, Claviceps, Tilletia, Phoma, Cochliobolus, Gaeumanomyces, Rhychosporium, Biopolaris, and Helminthosporium, and combinations thereof.
X4. The method according to any one of items X1-X3, wherein said one or more phytopathogen(s) is a species selected from the group consisting of Botrytis cinerea, Botrytis squamosa, Erwinia carotovora, Erwinia amylovora, Dickeya dadantii, Dickeya solani, Agrobacterium tumefaciens, Xanthomonas axonopodis, Xanthomonas campestris pv. carotae, Xanthomonas pruni, Xanthomonas arboricola, Xanthomonas oryzae pv. oryzae, Xylella fastidiosa, Candidatus liberibacter, Fusarium culmorum, Fusarium graminearum, Fusarium oxysporum, Fusarium oxysporum f. sp. Cubense, Fusarium oxysporum f. sp. Lycopersici, Fusarium virguliforme, Sclerotinia sclerotiorum, Sclerotinia minor, Sclerotinia homeocarpa, Uncinula necator, Podosphaera leucotricha, Podosphaera clandestine, Phomopsis viticola, Alternaria tenuissima, Alternaria porri, Alternaria alternate, Alternaria solani, Alternaria tenuis, Pseudomonas syringae pv. Tomato, Phytophthora infestans, Phytophthora parasitica, Phytophthora sojae, Phytophthora capsici, Phytophthora cinnamon, Phytophthora fragariae, Phytophthora ramorum, Phytophthora palmivara, Phytophthora nicotianae, Phakopsora pachyrhizi, Phakopsora meibomiae, Aspergillus flavus, Aspergillus niger, Uromyces appendiculatus, Cladosporium herbarum, Rhizopus arrhizus, Rhizoctonia solani, Rhizoctonia zeae, Rhizoctonia oryzae, Rhizoctonia caritae, Rhizoctonia cerealis, Rhizoctonia crocorum, Rhizoctonia fragariae, Rhizoctonia ramicola, Rhizoctonia rubi, Rhizoctonia leguminicola, Macrophomina phaseolina, Mycosphaerella graminocola, Mycosphaerella fijiensis, Mycosphaerella pomi, Mycosphaerella citri, Magnaporthe oryzae, Magnaporthe grisea, Monilinia fruticola, Monilinia vacciniicorymbosi, Monilinia laxa, Colletotrichum gloeosporiodes, Colletotrichum acutatum, Coletotrichum candidum, Diaporthe citri, Corynespora cassiicola, Gymnosporangium juniperi-virginianae, Schizothyrium pomi, Gloeodes pomigena, Botryosphaeria dothidea, Wilsonomyces carpophilus, Sphaerotheca macularis, Sphaerotheca pannosa, Stagonospora nodorum, Pythium ultimum, Pythium aphanidermatum, Pythium irregularum, Pythium ulosum, Pythium lutriarium, Pythium sylvatium, Venturia inaequalis, Ustilago nuda, Ustilago maydis, Ustilago scitaminea, Claviceps pupurea, Tilletia tritici, Tilletia laevis, Tilletia horrid, Tilletia controversa, Phoma glycinicola, Phoma exigua, Phoma lingam, Cochliobolus sativus, Gaeumanomyces gaminis, Rhychosporium secalis, Helminthosporium secalis, Helminthosporium maydis, Helminthosporium solani, and Helminthosporium triticirepentis, and combinations thereof.
X5. The method according to any one of items X1-X4, wherein said one or more phytopathogen(s) is Fusarium graminearum, Fusarium culmorum and/or Botrytis cinerea.
X6. The Bacillus amyloliquefaciens strain according to any one of items X1-X5, wherein said phytopathogen is Fusarium graminearum or Fusarium culmorum.
X7. The method according to any one of items X1-X6, wherein said phytopathogen is Fusarium graminearum.
X8. The method according to any one of items X1-X5, wherein said phytopathogen is Botrytis cinerea.
X9. The method according to any one of items X1-X5, wherein said one or more phytopathogens are Fusarium graminearum and Botrytis cinerea.
X10. The method according to any one of items X1-X9, wherein said parental strain of Bacillus amyloliquefaciens (FHB-P) is deposited as DSM34003 at Deutsche Sammlung von Mikroorganismen und Zellkulturen GmbH (DSMZ), Inhoffenstr. 7B, D-38124 Braunschweig, Germany, by Chr. Hansen A/S, Hørsholm, Denmark on 24 Aug. 2021.
X11. The method according to any one of items X1-X10, wherein said one or more mutagens are chemical mutagens and/or physical mutagens.
X12. The method according to item X11, wherein said one or more chemical mutagens are selected from ethyl methane sulphonate (EMS), alkylating agents (such as ethyl ethane sulphonate, methyl methane sulphonate, ethylene imines, etc.), base analogs, azides, and N-methyl-N′-nitro-N-nitroguanidine (MNNG), preferably EMS.
X13. The method according to any one of items X11 or X12, wherein the concentration of chemical mutagen is in the range of about 12-24 g/L mM, preferably in the range of about 18-24 g/L.
X14. The method according to any one of items X11-X13, wherein said one or more physical mutagens are selected from UV-light, gamma-rays and X-rays.
X15. The method according to any one of items X1-X14, wherein said inhibitory effect is determined by exposing said phytopathogen to a sample comprising a Bacillus amyloliquefaciens strain and measuring growth of said phytopathogen.
X16. The method according to item X15, wherein said sample further comprises one or more antibiotics.
X17. The method according to any one of items X15 or X16, wherein said growth is determined by measuring the optical density (OD) of said phytopathogen.
X18. The method according to any one of items X1-X17, wherein said one or more metabolites are lipopeptides.
X19. The method according to item X18, wherein said lipopeptides are selected from the group consisting of iturins, fengycins and surfactins, and combinations thereof.
X20. The method according to any one of items X1-X19, wherein said method further comprises a step (v) of sequencing and identifying single nucleotide polymorphisms (SNPs) of said one or more Bacillus amyloliquefaciens strains selected in step (iv).
P1. A Bacillus amyloliquefaciens strain obtainable by the method according to any one of items X1-X20.
Q1. A composition comprising:
   (i) a Bacillus amyloliquefaciens strain according to any one of items Y1-Y34 or P1, and/or
   (ii) a fermentation product produced by a Bacillus amyloliquefaciens strain according to any one of items Y1-Y34 or P1.
Q2. The composition according to item Q1, wherein said composition comprises the fermentation product produced by the Bacillus amyloliquefaciens strain.
Q3. The composition according to item Q1, wherein said composition comprises the Bacillus amyloliquefaciens strain.
Q4. The composition according to any one of items Q1-Q3, wherein said composition comprises:
   (i) the Bacillus amyloliquefaciens strain, and
   (ii) the fermentation product produced by the Bacillus amyloliquefaciens strain.
Q5. The composition according to any one of items Q1-Q4, wherein said fermentation product comprises increased levels of one or more metabolites compared to a fermentation product produced by said parental strain of Bacillus amyloliquefaciens, when cultured under the same conditions.
Q6. The composition according to item Q5, wherein said one or more metabolites are lipopeptides.
Q7. The composition according to item Q6, wherein said lipopeptides are selected from the group consisting of iturins, fengycins and surfactins, and combinations thereof.
Q8. The composition according to any one of items Q6 or Q7, wherein said lipopeptides are iturins and fengycins.
Q9. The composition according to any one of items Q1-Q8, wherein said composition comprises spores of said Bacillus amyloliquefaciens strain.
Q10. The composition according to any one of items Q1-Q9, wherein said composition further comprises one or more agrochemically acceptable excipients, carriers, surfactants, dispersants and yeast extracts.
Q11. The composition according to any one of items Q1-Q10, wherein said composition further comprises one or more active ingredients.
Q12. The composition according to item Q11, wherein said one or more active ingredients are of microbial, biological or chemical origin.
Q13. The composition according to any one of items Q11 or Q12, wherein said one or more active ingredients are selected from the group consisting of an insecticide, fungicide, nematicide, bactericide, herbicide, plant extract, plant growth regulator, a plant growth stimulator, and fertilizer.
Q14. The composition according to item Q13, wherein said insecticide is selected from the group consisting of pyrethroids, bifenthrin, tefluthrin, zeta-cypermethrin, organophosphates, chlorethoxyphos, chlorpyrifos, tebupirimphos, cyfluthrin, fiproles, fipronil, nicotinoids, and clothianidin, and combinations thereof.
Q15. The composition according to any one of items Q13 or Q14, wherein said fungicide is selected from the group consisting of fluopyram plus tebuconazole, chlorothalonil, thiophanate-methyl, prothioconazole, metalaxyl, and copper hydroxide, and combinations thereof.
Q16. The composition according to any one of items Q11-Q15, wherein said one or more active ingredients are selected from another strain of bacteria different from said Bacillus amyloliquefaciens strain.
Q17. The composition according to any one of items Q1-Q16, wherein said composition is a form selected from the group consisting of a liquid, a wettable powder, a granule, a spreadable granule, a wettable granule, a microencapsulation, and a planting matrix.
Q18. The composition according to any one of items Q1-Q17, wherein said composition is a liquid formulation.
Q19. The composition according to any one of items Q1-Q18, wherein said composition further comprises a coating polymer.
Q20. The composition according to any one of items Q1-Q19, wherein said compositions comprises two or more Bacillus amyloliquefaciens strains according to any one of items Y1-Y34 or P1.
Q21. The composition according to any one of items Q1-Q20, wherein said compositions comprises two Bacillus amyloliquefaciens strains according to any one of items Y1-Y34 or P1.
Z1. A method of inhibiting growth of one or more phytopathogens on a plant, said method comprising applying a Bacillus amyloliquefaciens strain according to anyone of items Y1-Y34 or P1 or a composition according to any one of items Q1-Q21 to the plant or a part of the plant.
Z2. The method according to item Z1, wherein said one or more phytopathogen(s) is Fusarium graminearum, Fusarium culmorum and/or Botrytis cinerea.
Z3. The method according to any one of items Z1 or Z2, wherein said phytopathogen is Fusarium graminearum or Fusarium culmorum.
Z4. The method according to any one of items Z1-Z3, wherein said phytopathogen is Fusarium graminearum.
Z5. The method according to any one of items Z1 or Z2, wherein said phytopathogen is Botrytis cinerea.
Z6. The method according to any one of items Z1 or Z2, wherein said one or more phytopathogens are Fusarium graminearum and Botrytis cinerea.
Z7. The method according to any one of items Z1-Z6, wherein the plant is selected from the group consisting of a crop, a monocotyledonous plant, a dicotyledonous plant, a tree, an herb, a bush, a grass, a vine, a fern, a moss, and green algae.
Z8. The method according to any one of items Z1-Z7, wherein the plant is selected from the group consisting of wheat, barley, oats, small cereal grains, corn, rice, sugar cane, soybean, potato, carrot, coffee and banana.
Z9. The method according to any one of items Z1-Z8, wherein the part of the plant is selected from the group consisting of a seed, fruit, root, stem, leaf, corm, tuber, bulb and rhizome.
Z10. The method according to any one of items Z1-Z9, wherein said Bacillus amyloliquefaciens strain or composition is applied to the foliage of the plant.
Z11. The method according to any one of items Z1-Z10, wherein said Bacillus amyloliquefaciens strain or composition is sprayed onto the plant.
V1. Use of a Bacillus amyloliquefaciens strain according to anyone of items Y1-Y34 or P1 or a composition according to any one of items Q1-Q21 for inhibiting growth of one or more phytopathogens on a plant.
V2. The use according to item V1, wherein said phytopathogens is Fusarium graminearum, Fusarium culmorum, and/or Botrytis cinerea.
V3. The use according to any one of items V1 or V2, wherein said Bacillus amyloliquefaciens strain or composition is applied to the foliage of the plant.
V4. The use according to any one of items V2 or V3, wherein inhibition of Fusarium graminearum or Fusarium culmorum growth facilitates improved seedling vigor, improved root development, improved plant growth, improved plant health, increased yield, improved appearance, reduced pathogenic infection, or a combination thereof.
V5. The use according to any one of items V1-V4, wherein said phytopathogens is Fusarium graminearum.
T1. A kit comprising:
   (i) a Bacillus amyloliquefaciens strain according to anyone of items Y1-Y34 or P1 or a composition according to any one of items Q1-Q21;
   (ii) a container; and
   (iii) optionally, instructions for use.
T2. The kit according to item T1, wherein the kit further comprises one or more active ingredients selected from the group consisting of an insecticide, fungicide, nematicide, bactericide, herbicide, plant extract, plant growth regulator, a plant growth stimulator, and fertilizer.
T3. The kit according to item T2, wherein said Bacillus amyloliquefaciens strain and said one or more active ingredients are provided in separate compartments in the container.

The invention will now be described in further details in the following non-limiting examples.

### EXAMPLES

### Example 1: Construction of EMS Libraries of Bacillus amyloliquefaciens

Libraries of a Bacillus amyloliquefaciens were prepared as part of a classical strain improvement strategy to produce derivatives of Bacillus amyloliquefaciens with improved fusarium head blight inhibition.

### Method:

### Media and Growth Condition

Bacillus strains were cultured in LB liquid or solid agar media at 37 °C and 250 RPM.

CFU Count of Bacillus amyloliquefaciens Strain

An overnight saturated culture of Bacillus amyloliquefaciens FHB-P was freshly inoculated in 5 ml of LB and incubated at 37 °C and 250 RPM. At the optical density (OD₆₀₀ₙₘ) 1, the culture was 10-fold serially diluted from 10⁻¹ to 10⁻⁷ and 100 μl of the serially diluted cultures were spread in LB agar plate. The plates were incubated overnight at 30 °C and next morning colonies appearing in the plates were counted. The number of colonies in the plate were used for determining CFU/ml using equation I:

$$CFU / ml = \frac{n umber of colonies \times dilution factor}{volume of culture plated ( μl )} \times 100 \qquad ( equation I )$$

It was determined that 1 ml of 1.0 OD₆₀₀ₙₘ of fermentation product of Bacillus amyloliquefaciens FHB-P contains 1.76×10⁸ viable cells.

Toxicity Test and MIC Determination of Ethyl Methanesulfonate (EMS) for Bacillus amyloliquefaciens Strain

The effective concentration of EMS that is required to mutate the Bacillus strains is a basic requirement for the induced mutagenesis process. First, an EMS toxicity test was performed to find the minimum inhibitory concentration (MIC) of EMS for the Bacillus amyloliquefaciens strain under laboratory condition.

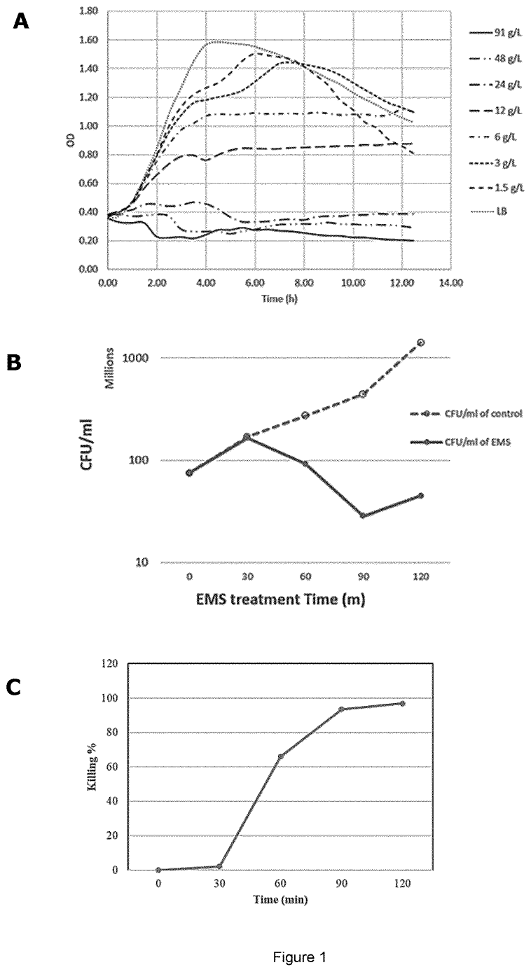

Bacillus amyloliquefaciens FHB-P was inoculated in LB media and overnight incubated at 37 °C and 250 RPM. Then next morning, the saturated cultures were inoculated in LB media supplemented with various concentration (from 0 to 91 g/L) of EMS with initial culture OD₆₀₀ₙₘ of 0.1. Then the Bacillus growth was monitored in every 30 min using Growth-profiler. The MIC of EMS for Bacillus amyloliquefaciens FHB-P was determined by analysing the obtained growth curves (FIG. 1A) and the growth profile as a function of EMS concentrations was used to decide which EMS concentrations to use for construction of the mutagenesis libraries.

Determination of Killing Rate Percentage of Bacillus amyloliquefaciens by EMS

An EMS concentration of 18 g/L was selected for determining killing rate percentage as this strain can grow at 12 g/L of EMS but not at 24 g/L (FIG. 1A).

In eppendorf tubes, 1 ml of Bacillus amyloliquefaciens FHB-P culture of OD600 nm 1 were taken. Then 18 g/L of EMS was added in one tube whereas the culture in only LB media was taken as control. The eppendorf tubes were incubated in thermo-block at 37 °C and 700 RPM. At 0 min, 30 min, 60 min, 90 min and 120 min, 100 μl of the cultures were withdrawn. The samples were 10-fold serially diluted from 10⁻¹ to 10⁻⁷ and 100 μl of the diluted samples were plated on LB agar plates. The plates were incubated overnight at 30 °C

Next morning, colonies forming in the LB agar plates were counted and CFU/ml were calculated. Using these CFU/ml values, killing rate percentage at each time point were calculated using equation II:

$$Killing rate \% = \frac{\begin{matrix}CFU / ml of control culture - \\ CFU / ml of EMS treated culture\end{matrix}}{CFU / ml of control culture} \times 100 \% \qquad ( equation II )$$

After determining the killing rate %, the random mutagenesis libraries were constructed using selected concentrations of EMS and EMS treatment time aiming at a desired killing rate % of the library to control the degree of target mutations.

Construction of EMS Libraries of Bacillus amyloliquefaciens

In multiple eppendorf tubes, 1.5 ml of the exponentially growing Bacillus amyloliquefaciens FHB-P (with OD₆₀₀ₙₘ 1), EMS was added in concentrations of 18 g/L or 21.7 g/L. Then the cell cultures with EMS were incubated at 37 °C and 700-750 RPM in thermo-block. At 0, 30, 60, 90, 120 and 165 min of EMS exposure time, 100 μl of the cultures were serially diluted in pepsal from 10⁻¹ to 10⁻⁶ fold. Then 100 μl of the diluted samples were plated in LB agar and the plates were incubated overnight at 30 °C for determination of killing rate %. The remaining 1400 μl of the EMS treated culture was centrifuged at 5000-6000×g for 3 min and the cell pellet was washed twice in LB media to remove EMS and finally the cell pellet was resuspended in 25 ml of LB media and grown overnight at 30 °C

Next morning, colonies were counted in the plates incubated overnight to determine CFU/ml and killing rate % as described in equation I and II above.

### Results:

Growth curves of Bacillus amyloliquefaciens FHB-P cultured at increasing concentration of EMS showed that this Bacillus strain proliferate at an EMS concentration of 12 g/L but not at 24 g/L (FIG. 1A). Therefore, an EMS concentration between these two outer points can be used to induce mutations in the strain.

The growth rate and killing rate percentage was assessed over time to determine temporal conditions at which a high percentage of bacteria are killed (deselected) and at the same time some mutated and prolific derivatives are obtained.

To this end, the CFU/ml for the control culture and EMS treated cultures were plotted over the time (FIG. 1B). After addition of 18 g/L of EMS, in the first 30 min the CFU/ml was the same as that of the control sample. However, after 30 min the CFU/ml of EMS treated culture dropped whereas in case of the control the CFU/ml linearly increased. After 90 min, the CFU/ml started to increase slowly for the EMS treated samples indicating that the survival cells have gained EMS tolerance.

Determination of the killing rate percentage showed that at around 120 min of EMS treatment (using 18 g/L) a killing rate of 96% was achieved for Bacillus amyloliquefaciens FHB-P (FIG. 1C). To increase the killing rate percentage over 99%, the random mutagenesis libraries of Bacillus amyloliquefaciens FHB-P were prepared by using 18 g/L and 21.7 g/L of EMS.

In total four EMS libraries were constructed;
1. EMS-Library-1 constructed by using 18 g/L of EMS and 165 min exposure time
2. EMS-Library-2 constructed by using 18 g/L of EMS and 120 min exposure time
3. EMS-Library-3 constructed by using 21.7 g/L of EMS and 165 min exposure time
4. EMS-Library-4 constructed by using 21.7 g/L of EMS and 120 min exposure time

The CFU/ml counts and the killing rate % of the final libraries are given in Table I. About 99.6% to about 99.9% of the cells were killed by the supplemented EMS over the exposure time of 120 min to 165 min.

TABLE I
CFU/ml and killing rate % of the EMS libraries
of Bacillus amyloliquefaciens FHB-P.

| | Library no. | | | | Parental |
|---|---|---|---|---|---|
| | 1 | 2 | 3 | 4 | (control) |
| EMS conc. (g/L) | 18 | 18 | 21.7 | 21.7 | — |
| Exposure time (min) | 165 | 120 | 165 | 120 | — |
| Cell count (CFU/ml) | 1.00E+05 | 1.00E+06 | 9.00E+05 | N/A | 7.70E+08 |
| Killing rate (%) | 99.96 | 99.57 | 99.61 | N/A | — |

### Conclusion

This example demonstrates that libraries were successfully designed for preparation of mutated and prolific derivatives of Bacillus amyloliquefaciens FHB-P.

### Example 2: Screening of EMS Libraries for Derivatives of Bacillus amyloliquefaciens with Increased Inhibitory Effect on Fusarium graminearum

Libraries of a Bacillus amyloliquefaciens were screened to identify derivatives of Bacillus amyloliquefaciens with improved fusarium head blight inhibition.

### Method:

### Media and Growth Condition

The Fusarium strains were cultivated in mung bean media with 16 h of light and 8 h of dark cycles for spore production and potato dextrose broth (PDB) liquid media and potato dextrose agar (PDA) solid media for inhibition assays at 28 °C

PDB with Fusarium spores (for automated screening):
500 ml 2×PDB (in a 1 L bottle), 495 ml Pepsal, 2.5 ml 10 mg/ml Chloramphenicol, 5 ml Fusarium stock (3×10⁵ spores Fusarium per ml)
Final spore concentration: 1.5×10³ spores/mL

For the fungal inhibition assay, Bacillus strains were cultivated in LB, M2, or MSgg.

### M2 Medium:

Peptone (J636, Amresco, 10 g/L), Yeast extract (LP0021, Oxoid, 6 g/L), MgSO₄—7H₂O (M1880, Sigma-Aldrich, 0.5 g/L), KCl (P9541, Sigma, 2 g/L), KOH to adjust pH to 7.0. The following is added filtered; 1M Ca(NO₃)₂ (C1396, Sigma, 1 ml/L), 0.1M MnCl₂ (M5005, Sigma, 1 ml/L), 10 mM FeSO₄ (215422, Sigma, 100 μl/L), 20% Glucose (G8270, Sigma, 5 ml/L)

### MSgg Medium:

5 mM potassium phosphate (pH 7), 100 mM MOPS (pH 7), 2 mM MgCl₂, 700 mM CaCl₂, 50 mM MnCl₂, 50 mM FeCl₃, 1 mM ZnCl₂, 2 mM thiamine, 0.5% glycerol, 0.5% glutamate.

### Fungi Spore Counts and Spore Stock Preparation

About 1-2 cm² sized agar plug containing freshly grown Fusarium graminearum strains in PDA plates were added in 200 ml of 4 g/L and 10 g/L of mung bean media in a 500 ml culture flask for preparation of spores. The flasks were incubated at 28 °C with 16 h of light and 8 h of dark cycles for 14 days. The spore counts were performed by using 22 μl of the culture broth in the glass slides with a grid of 10 squares, each square having dimensions of 1×1 mm, a depth of 0.1 mm and a volume of 0.1 μl. The concentration or number of spores/ml were determined using equation III:

$$Spores / ml = ( \sum spores counted in square N ) \times \frac{dilution factor \times {10}^{4}}{N} \qquad ( equation III )$$

The Fusarium graminearum cultures were filtered through sterile mira-cotton clothes to remove fungal mycelia and other insoluble media components. The filtrate was centrifuged at 4500×g for 10 min and supernatant was discarded. Finally, the spores were resuspended in Saline-tween (8 g/L of NaCl and 50 μl/L of Tween 80) with 25% of glycerol and spore concentration was determined after 10-fold and 100-fold dilutions using equation III. The spore stocks were aliquoted in eppendorf tubes and stored at −80 °C

### Fusarium Graminearum Spore Germination Test

A frozen spore stock (3×10⁵ spores/ml concentration) was thawed in ice-water bath and then the spore was serially diluted in PDB+Cm (25 μg/ml) media from 10⁻¹ to 10⁻⁸. Two hundred microliters of the diluted spores in PDB media were taken in 96 well microtiter plate (in six replicates for each dilution). The microtiter plate was covered with the lid and sealed with 3M tape. Then the plate was incubated at 25 °C Optical density at 600 nm was measured in every 24 h to analyze the fungal spore germination and growth.

Manual Screening of Bacillus amyloliquefaciens EMS Libraries for Improved Fusarium Inhibition

For manual screening, three EMS libraries of FHB-P (library nos. 1, 2 and 3) were selected.

For manual fungal inhibition assay, 10 spores of Fusarium graminearum in 200 μl of PDB media supplemented with 25 μg/ml of chloramphenicol were taken in each well of a microtiter plate. The Bacillus amyloliquefaciens FHB-P and the isolates of libraries were cultivated in LB media at 37 °C and 250 RPM for 24 h. Then, 7.5 μl, 10 μl and 13 μl of the fermentation product were added in the microtiter plate containing Fusarium spores in PDB+Cm media. The plates were sealed with 3M tape and then incubated at 25 °C The plates were evaluated by visual inspection and OD measurements were performed on day 3-6.

For further validation of the improved fungal inhibition activities, selected Bacillus amyloliquefaciens derivatives (hits with improved fungal inhibition activity) were inoculated in 400 μl of LB media in 96 deep well plates in quadruplets and eight replicates of the parental Bacillus strain and four wells with only LB media (without inoculation) were also included in the same plate and the plates were incubated at 37 °C and 250 RPM for 24 h. Glycerol stocks (final glycerol concentration of 20%) of the saturated cultures were prepared by taking 100 μl of the fermentation product. The remaining cultures in the deep well plates were centrifuged at 3700 RPM for 30 min at room temperature. Then 100-150 μl of the fermentation product were carefully transferred into sterile microtiter plate. Then the Fusarium inhibition assay was repeated as described above using 5 μl, 7.5 μl, 10 μl and 13 μl of fermentation products of selected Bacillus amyloliquefaciens improved hits in quadruplets.

Automated High-Throughput Screening Bacillus amyloliquefaciens EMS Libraries for Improved Fusarium Inhibition

For automated screening, two EMS libraries (library nos. 3 and 4) were selected.

The automated screening of the mutant libraries was performed in a total of four batches, each of 4048 mutants per week (44×92), following a 3-step method as described below.

### Colony Picking and Liquid Culture of Mutants:

Large square LB agar plates (Q-trays) containing mutant colonies were processed in a Qpix2 colony picker. The colonies were deposited into 96w-MTPs containing 200 μl of M2 media and were grown overnight at 34 °C with 260 RPM shaking in a stroke incubator. The positive control (parental strain FHB-P and blank were added on the last 4 wells of each plate. The plates were covered with the Aeraseal/Breatheasier paper covers that allow for better oxygenation than plastic lids. Furthermore, the plates in a stack were spaced with clean tips for improved air accessibility and incubated at 25 °C

### Biomass Pelleting and Fermentation Product Dilution:

The screening aims to find mutant strains with inhibition potential based on the secreted compounds. Thus, only the fermentation product is to be tested against the Fusarium growth. As the parental strain, most of the mutants were strong biofilm producers. Thus, the plates were centrifuged for at least 20 min at >3000×g. All strains were subsequently cryo-stored at −80 °C after addition of glycerol media to a final glycerol concentration of 20%.

### Inhibition Assay:

The fermentation product was diluted to the desired concentration (41-fold dilution). This was performed by adding 5 μl of fermentation product into 200 μl of Pepsal. 50 μl of this dilution were added to 200 μl of PDB with Fusarium. The plates were then covered with Breath-Easy transparent oxygen permeable seals and incubated at 25 °C, without stacking to allow enough oxygenation for all wells. OD₆₂₀ₙₘ was used to quantify Fusarium growth, and all the plates were measured twice, first at T=20 h and after 90h. The difference between these two measurements, normalized to the average value of the blanks was used as a hit selection criterion. The hit designation was awarded if the growth values were at least 2 standard deviations lower than those of the parental strain. Wells with an initial high OD measurement (time 20h, OD>0.3) were discarded, due to the high possibility of biofilm transfer during the dilution process.

### Results:

Fusarium graminearum Spore Germination Test

A Fusarium graminearum strain with 5.2×10⁴ spores/ml was prepared as a spore glycerol stock for manual screening of fungal inhibition capabilities. For automated screening, a Fusarium graminearum strain with 3.0×10⁵ spores/ml was prepared.

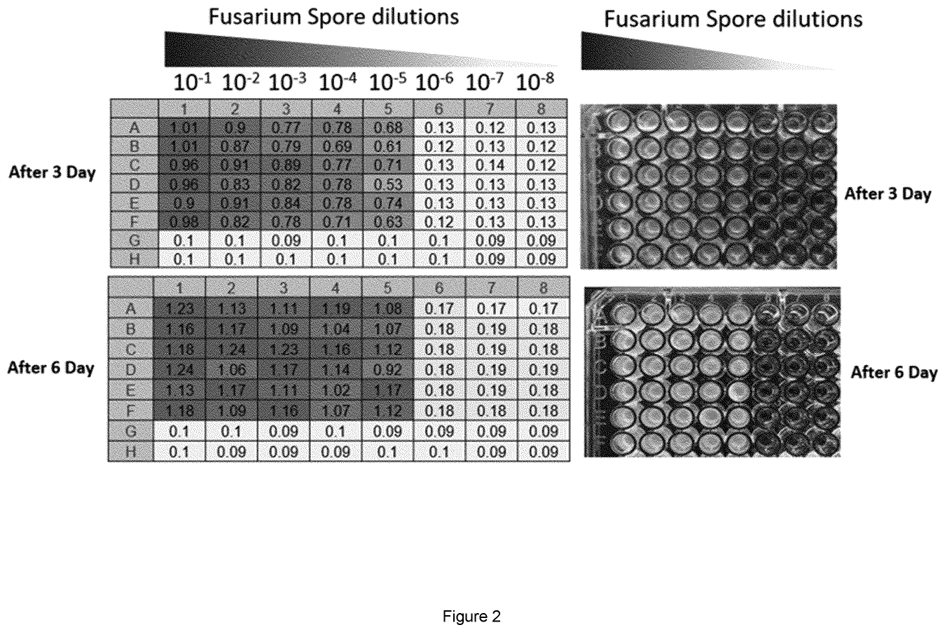

From the growth observation (both OD reads and plate observation, FIG. 2), it is clear that after 3 days of incubation, there is homogeneous growth of the Fusarium up to 10⁻⁴ fold diluted spores and after 6 days of incubation fusarium growth was saturated up to 10⁻⁵ fold diluted spores. Hence, for the spore stock with 3×10⁵ spores/ml concentration, 10⁻⁴ fold dilution is optimal to obtain the fusarium growth in 3 day and based on this observation a 10⁻³ fold dilution was chosen for manual screening using a spore stock of 5.2×10⁴ spore/ml concentration.

### Manual Screening of Libraries for Improved Antifungal Activity

To select the improved hits from the EMS libraries of Bacillus amyloliquefaciens_FHB-P, single colonies (isolates) were inoculated in 150 μl of LB media in 96 microtiter plates. Three to four replicates of the parental strain Bacillus amyloliquefaciens FHB-P were also inoculated in the same plate (as a control). In the manual screening total 2332 isolated were picked from three different EMS libraries: 1134 isolates from the library 1, 929 isolates from library 2 and 269 isolates from library 3.

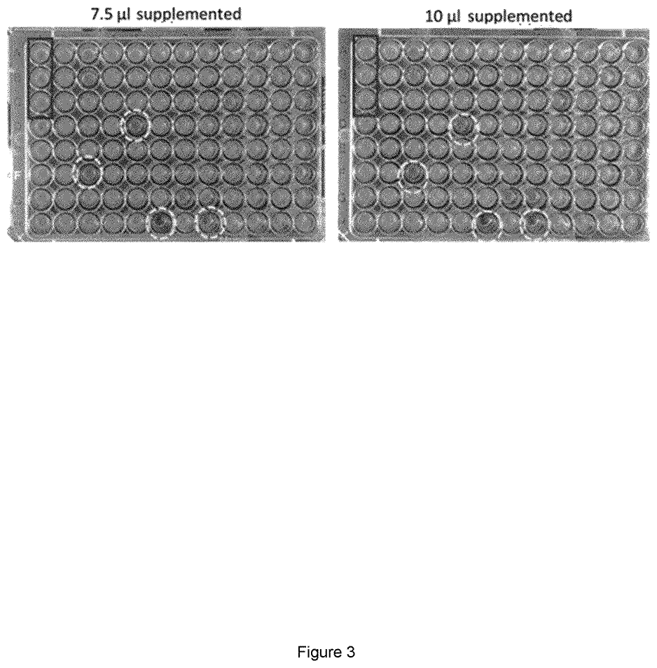

The inhibition assay was carried out as described above. The plates supplemented with various amount of Bacillus fermentation products in diluted fungal spores in PDB+Cm were observed for Fusarium growth and all the wells with less or no growth of Fusarium as compared to the corresponding control well were selected as hits (see FIG. 3 for example). A total of 84 hits (34 hits originated from library 1, 23 hits originated from library 2 and 27 hits originated from library 3) were selected for further validation to reconfirm the improved antifungal activity as described above.

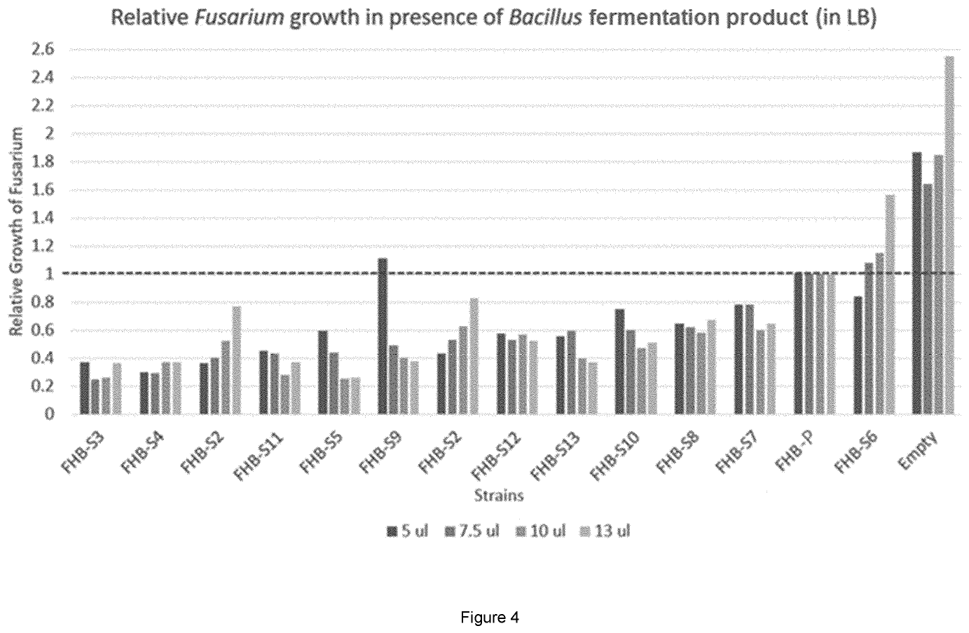

To rank the improved strains, the relative Fusarium inhibition by the improved hits as compared to the inhibition offered by parents was plotted. 25 hits (originating from libraries 1 and 3) displayed improved Fusarium inhibition activity as compared to the parental strain, out which 13 were selected as main lead strains from the manual screening. The relative Fusarium inhibition offered by the selected 13 lead strains are depicted in the FIG. 4. Among the selected leads, about 50-60% improved Fusarium inhibition was displayed by FHB-S3, FHB-S4, FHB-S1, FHB-S11, FHB-S9 and FHB-S2.

### Automated High-Throughput Screening of Libraries for Improved Antifungal Activity

Selection by automated screening followed the description above with hit designation being awarded if the Fusarium growth values were at least 2 standard deviations lower than those of the parental strain.

A total of 87 hits were defined and tested more stringently for their activity. The selected hits were re-tested at multiple dilution levels of the fermentation product (5× to 320×, with 2 fold steps) and having 2 replicates per sample. Using the data from the different dilution levels, it was possible to estimate an ID50 (50% inhibition of the maximal fungal growth) value and select those derivative strains better than the parental strain. A total of 31 lead hits displayed favourable ID50 values.

### Conclusion

This example demonstrates that out of 2332 isolates, manual screening identified 25 improved Bacillus strains with improved Fusarium graminearum inhibition. 13 lead strains were selected therefrom, some of which displayed 50-60% improved Fusarium graminearum inhibition.

The example also demonstrates that automated high-throughput screening of Bacillus amyloliquefaciens EMS libraries successfully identified 31 lead strains with improved ID50 values.

### Example 3: Characterization of Selected Lead Derivatives of Bacillus amyloliquefaciens

Selected lead derivatives of Bacillus amyloliquefaciens with desired traits were further characterized to determine the most improved candidate strains.

### Method:

### Media and Growth Condition

### Bacillus Sporulation Medium:

8 g/L of Difco Nutrient Broth, 1 g/L of KCl, 0.25 g/L of MgSO₄ 7H₂O, 4.4 mg/L of (NH₄)₅[Fe(C₆H₄O₇)₂], 10 μM of MnCl₂, and 500 μM of CaCl₂.

### Sporulation Test

Derivative strains of Bacillus amyloliquefaciens with increased Fusarium growth inhibition activities identified in Example 2 were tested for sporulation using microscopic analysis and a heating method.

In the microscopic analysis method, the selected improved derivative strains of Bacillus amyloliquefaciens were cultivated in M2 or LB media to saturation (from 18-24 h). Then 1 μl of the culture was analyzed under microscope with 100× magnification. If spores were visible, then they were categorized as a spore positive strain and if the spores were not visible in microscope, then those strains were further tested using the heating method.

For the heating method, the selected improved derivative strains of Bacillus amyloliquefaciens were cultivated overnight in sporulation media at 37 °C and 250 RPM.

Then 50 μl of the overnight cultures were taken into PCR tubes and heated in the tubes at 80 °C for 20 min. The heat-treated cultures were serially diluted from 10⁰-10⁻⁷ and 5 μl of the diluted cultures were spotted in LB agar plate. The spore positive strain (parent strain) and spore negative strain were also included in the assay as positive and negative controls. The LB agar plates were incubated overnight at 30 °C and next morning the spots were observed for the growth of Bacillus. If growth were observed in the heat-treated samples, those samples were categorized as spore positive strains.

Biochemical Analysis of Derivatives of Bacillus amyloliquefaciens

Single colonies of derivative strains of Bacillus amyloliquefaciens were inoculated in 600 μl of LB media in 96 deep well plate in triplicates and the plate was incubated at 30 °C for 24 h. Then the fermentation products were submitted for quantification of lipopeptides (iturins, fengycin and surfactins families).

For the lipopeptide analysis of the Bacillus strains in MSgg media, a saturated overnight culture of the strain in LB media was inoculated in 100 ml of MSgg media with a starting OD₆₀₀ₙₘ of 0.1. Then the flask was incubated at 37 °C and 250 RPM for 48 h followed by submission of 500 μl of the fermentation product for lipopeptide analysis. Lipopeptide samples were analyzed by high resolution mass spectrometer on a Quadrupole Time of Flight mass spectrometer connected to an ultra-high performance liquid chromatography separation unit.

### Fusarium Inhibition Activity

The Fusarium inhibition activity of all 44 lead strains was tested according to example 2 by growing Bacillus cultures in three different media; LB medium, MSgg medium, and M2 medium. Fermentation products obtained were tested and compared for their bioactivity against Fusarium.

### Whole Genome Sequencing

A single colony of the validated leads of the improved derivative strains of Bacillus amyloliquefaciens were purified by passing three rounds in LB plates and then 5 colonies were inoculated in 5 ml of LB media and the tube was incubated overnight at 30 °C for 250 RPM. Next morning, 500 μl of the saturated cultures were transferred in 30 ml of LB media in a flask which was incubated at 37 °C for 250 RPM. At around 1.5-2.0 OD₆₀₀ₙₘ of the cultures, whole genome sequencing samples were prepared.

For the whole genome sequencing, cell pellets from 1.5 ml of 1.5 OD equivalent cultures were collected by centrifuging the cultures at 5000×g for 10 min. Then the cell pellet was submitted for gDNA isolation and whole genome sequencing.

### Results:

### Sporulation Test

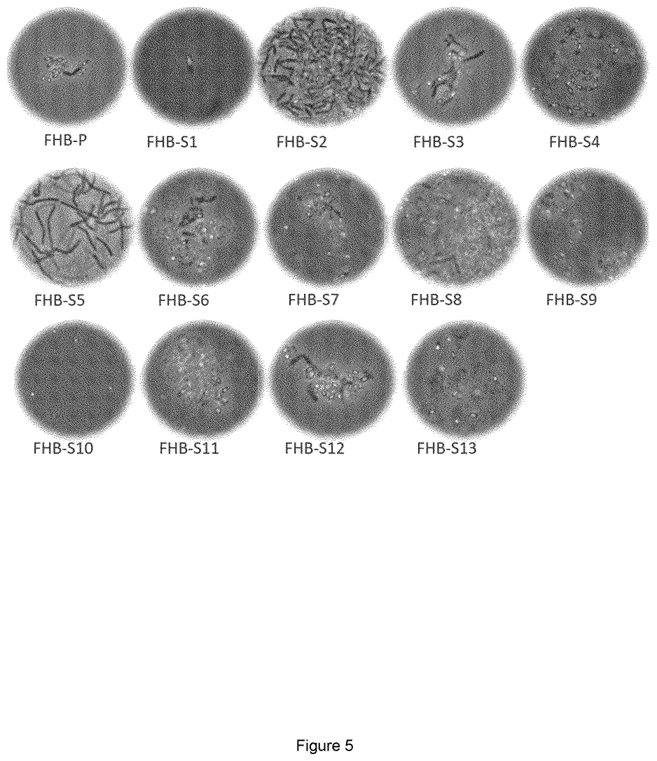

Both vegetative cells as well as spores were observed for all the selected lead strains (from both automated and manual screening), with the exception of FHB-S5 (sporulation test of manually screened hits are shown in FIG. 5). This observation was confirmed by all non-sporulating strains being killed during heat treatment.

### Production of Antifungal Lipopeptides; Iturin, Fengycin and Surfactin

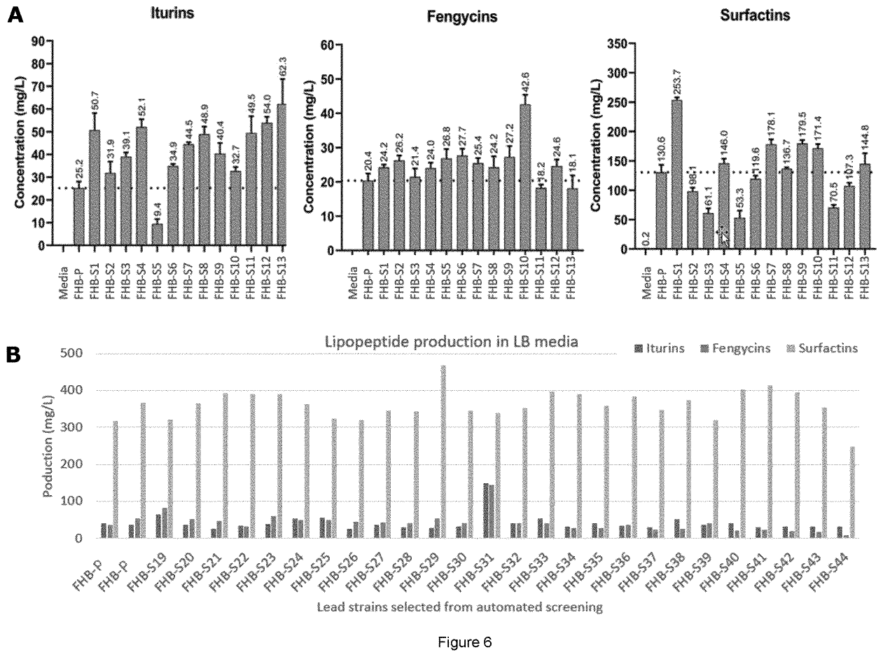

Overall, most of the lead strains selected from the manual screening produced significantly increased amount of lipopeptides as compared to the parent control (FIG. 6A). Especially production of iturin and fengycin was increased. For the lead strains selected by automated screening, particularly the strain FHB-S31 had increased production of iturin and fengycin (FIG. 6B). Both of these lipopeptide production assays were performed in LB media, whereas only the manual screening was performed in LB media (the automated screening was in M2 media).

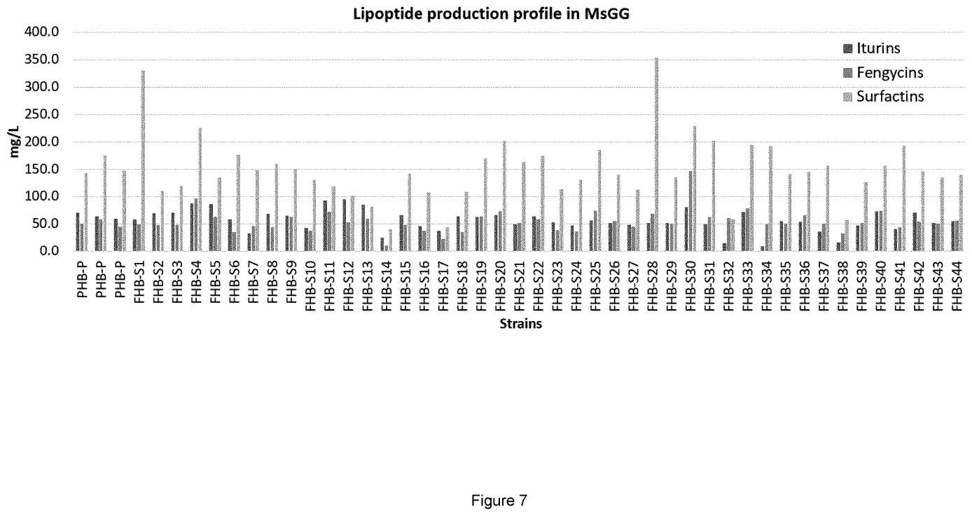

Lipopeptide analysis was also performed in MSgg media (FIG. 7). The experiment was completed in three batches of cultures and in each batch the parental strain was included as control. Therefore, the parental strain is displayed three times in FIG. 7. Notably, the improved lead strains FHB-S31 and FHB-S4 produced increased amounts of all three lipopeptides as compared to the parental strain.

### Fusarium Inhibition Activity

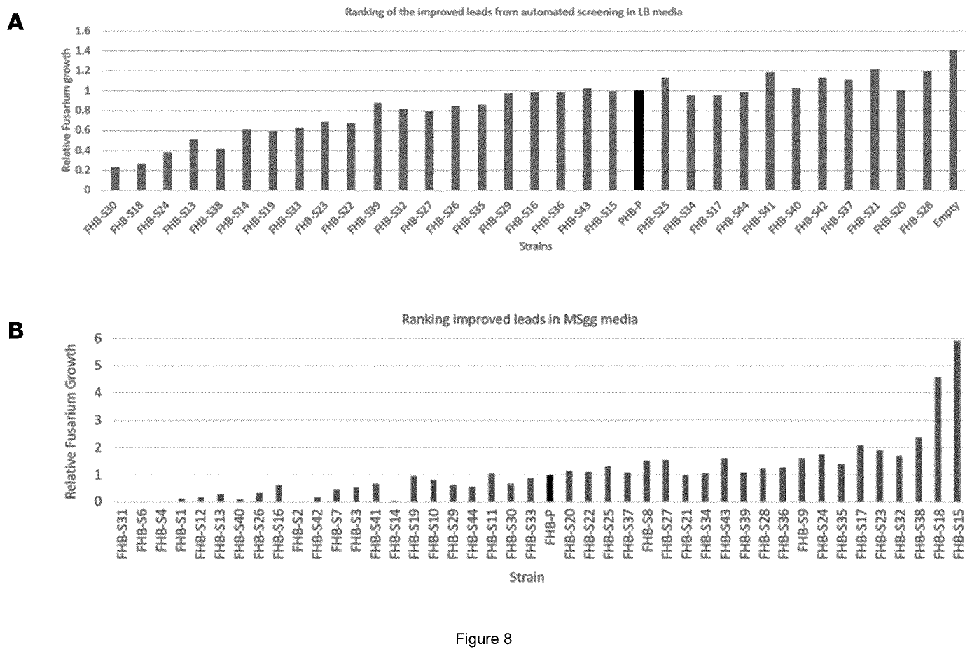

Inhibition capability of the derivative strains of Bacillus amyloliquefaciens were determine in different culture media. The results were scored as the relative ability to inhibit Fusarium growth compared to the parental Bacillus amyloliquefaciens strain FHB-P. The results for LB media (FIG. 8A), MSgg media (FIG. 8B), and M2 media (data not shown) all show that many derivative strains of Bacillus amyloliquefaciens with improved inhibition of Fusarium were identified, i.e. the relative Fusarium growth is less than 1 (wherein the parental strain equals 1).

### Whole Genome Sequencing/SNPs Analysis of the Improved Lead Strains

All 44 lead strains were whole genome sequenced. It was found that the number of mutations varied among the lead strains ranging from 6 to 44. All of the lead strains have large number of unique mutations and less numbers of common/shared mutations. The larger number of unique mutations might belong to the random mutations (non-beneficial to the target phenotypes) as all of these strains were generated by treatment with EMS mutagen. Since all of these strains have improved Fusarium inhibition activities, many of the mutations might have a same/common target in lipopeptide biosynthesis or regulation.

### Conclusion

This example demonstrates that a selection of derivative strains of Bacillus amyloliquefaciens with improved inhibition of Fusarium graminearum could be prepared. The most improved derivative strains produced highly increased levels of lipopeptides.

### Example 4: Inhibition of Fusarium graminearum by a Selection of Lead Derivative Strains of Bacillus amyloliquefaciens—ID50 and Lipopeptide Production

A selection of the most promising candidate strains was selected for upscaling and further characterization to link the changes in genotype to the improved inhibition of phytopathogens.

### Method:

Seven derivates of Bacillus amyloliquefaciens FHB-P (FHB-S31, FHB-S14, FHB-S19, FHB-S2, FHB-S1, and FHB-S11) were grown in an industrially suitable medium for bioactive metabolite production and the fermentation product at the end of the fermentation was used for in vitro inhibition experiments and quantification of metabolites.

As described in example 2, the dilution factor at which each strain derivative inhibits 50% of the maximal fungal growth (ID50) were used as a measure of inhibition potency. ID50 values were determined by sigmoid regression of experimental data obtained from in vitro fungal inhibition assays with different dilution factors. Experiments were done as biologically independent duplicates and results represent the averages and standard deviations calculated from results.

Bioactive metabolites, such as lipopeptide compounds of the three main families (iturins, fengycins and surfactins), were quantified by liquid chromatography-mass spectrometry (LC-MS) as described in example 3.

Following determination of ID50 values and lipopeptide production, the genome information obtained as described in example 3, were analyzed for each of the seven derivatives (FHB-S31, FHB-S14, FHB-S19, FHB-S2, FHB-S1, and FHB-S11).

### Results:

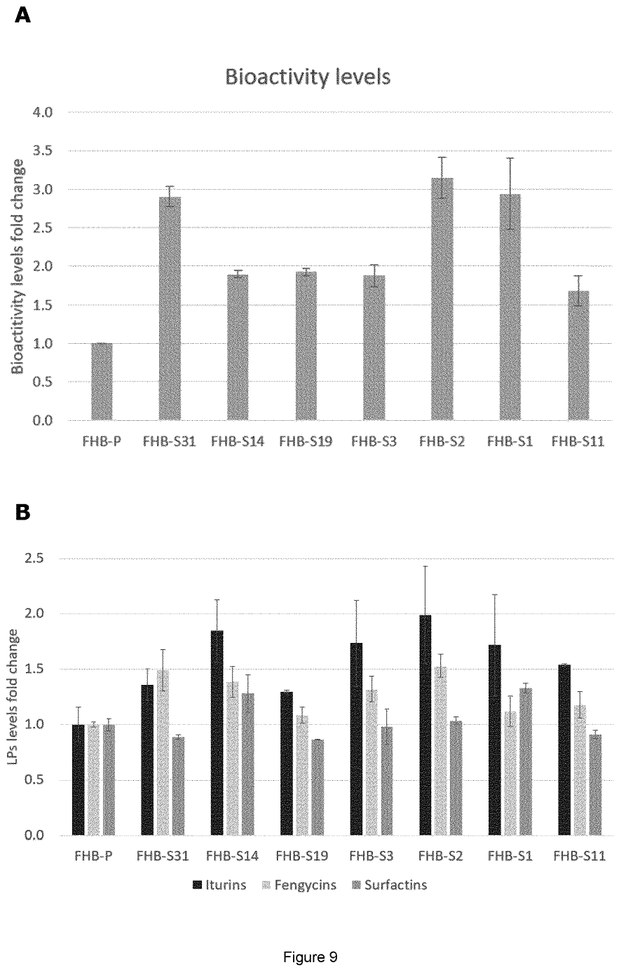

The data confirmed that the genetic changes accumulated in the derivative strains result in an increased fungal inhibitory potential. FIG. 9A shows the bioactivity level displayed as the fold change in ID50 values of the derivative strains normalized to the ID50 value of the parental strain (FHB-P). In all seven derivatives ID50 values were significantly above the parental strain, with bioactivity fold changes between 1.5 and 3.

Levels of metabolites produced by each strain derivative and the parental strain were compared and the results confirmed increased production of lipopeptides (FIG. 9B). Particularly, levels of both iturins and fengycins were increased for all improved derivatives.

Strain derivatives carrying genetic changes in the proteasome components (i.e. ClpP or ClpC) (FHB-S31, FHB-S14, and FHB-S1) or the ATP-dependent zinc metallopeptidase FtsH (FHB-S2) showed the highest bioactive metabolite levels, corresponding to the highest bioactivity levels. In a second group, derivatives with genetic changes targeting the alternative sigma factor SigH (FHB-S14, FHB-S19 and FHB-S11) also showed levels of metabolites and bioactivity above the parental strain.

### Conclusion

This example demonstrates that derivatives strains of Bacillus amyloliquefaciens FHB-P with improved potency to inhibit the growth of Fusarium spp. has been identified. Biochemical analysis of the fermentates confirmed increased bioactive metabolite production (iturins, fengycins and surfactins) of the improved derivatives when compared to the parental strain. Genome sequence analysis allowed identification of the genetic changes accumulated in the improved derivatives. Correlation between specific genetic changes within a few target genes and the improved phenotype for fungal inhibition has been established.

### Example 5: Inhibition of Botrytis cinerea by a Selection of Lead Derivative Strains of Bacillus amyloliquefaciens—ID50 and Lipopeptide Production

A selection of the most promising candidate strains was selected for upscaling and further characterization to link the changes in genotype to the improved inhibition of Botrytis cinerea.

### Method:

Ten derivatives of Bacillus amyloliquefaciens FHB-P (FHB-S21, FHB-S11, FHB-S09, FHB-S01, FHB-S44, FHB-S14, FHB-S43, FHB-S07, FHB-S17 and FHB-S08) were grown in M2 medium for bioactive metabolite production and the fermentation product at the end of the fermentation was used for in vitro inhibition experiments and quantification of metabolites.

The dilution factor at which each strain derivative inhibits 50% of the maximal fungal growth (ID50) were used as a measure of inhibition potency. ID50 values were determined by sigmoid regression of experimental data obtained from in vitro fungal inhibition assays with different dilution factors. Experiments were done as triplicates and results represent the averages and standard deviations calculated from results.

Bioactive metabolites, such as lipopeptide compounds of the three main families (iturins, fengycins and surfactins), were quantified by liquid chromatography-mass spectrometry (LC-MS) as described in example 3.

Following determination of ID50 values and lipopeptide production, the genome information obtained as described in example 3, were analysed for each of the ten derivatives (FHB-S21, FHB-S11, FHB-S09, FHB-S01, FHB-S44, FHB-S14, FHB-S43, FHB-S07, FHB-S17 and FHB-S08)

### Results:

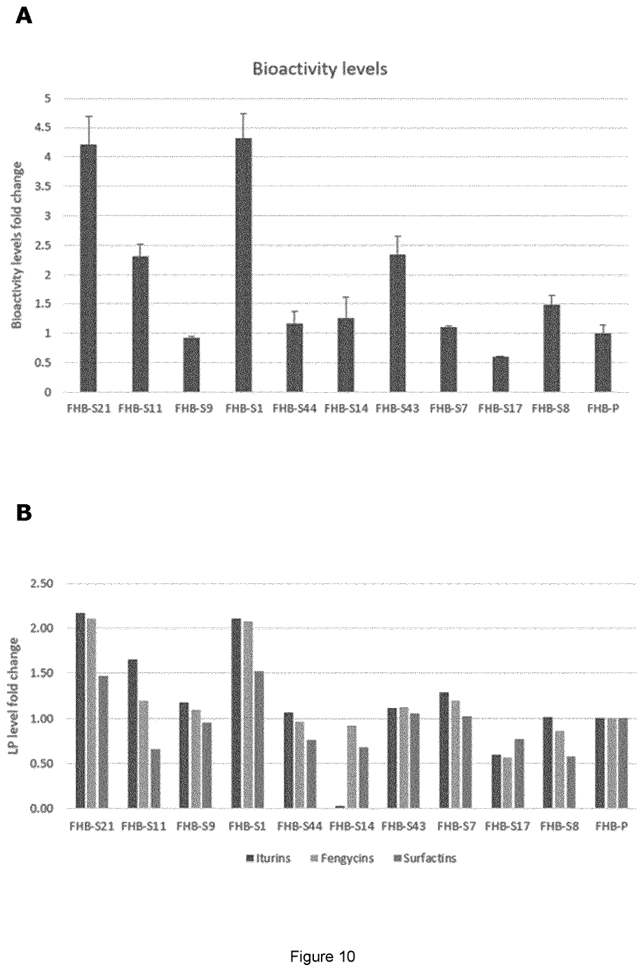

The data confirmed that the genetic changes accumulated in the derivative strains result in an increased fungal inhibitory potential. FIG. 10A shows the bioactivity level displayed as the fold change in ID50 values of the derivative strains normalized to the ID50 value of the parental strain (FHB-P). In the best derivatives, ID50 values were significantly above the parental strain, with bioactivity fold changes between 1.2 and 4.5.

Levels of metabolites produced by each strain derivative and the parental strain were compared and the results confirmed differential production of lipopeptides (FIG. 10B).

### Conclusion:

This example demonstrates that derivatives strains of Bacillus amyloliquefaciens FHB-P with improved potency to inhibit the growth of Botrytis spp. have been identified. Biochemical analysis of the fermentates confirmed increased bioactive metabolite production (iturins, fengycins and surfactins) of the improved derivatives when compared to the parental strain. Genome sequence analysis allowed identification of the genetic changes accumulated in the improved derivatives. Correlation between specific genetic changes within a few target genes and the improved phenotype for fungal inhibition has been established.

### REFERENCES

Altschul et al. (1990), J. Mol. Biol., 215, 403-410

### Deposits and Expert Solution

The applicant requests that a sample of the deposited microorganisms stated in table II below may only be made available to an expert, until the date on which the patent is granted.

The applicant requests that the availability of the deposited microorganism referred to in Rule 33 EPC shall be effected only by the issue of a sample to an independent expert nominated by the requester (Rule 32(1) EPC). If an expert solution has been requested, restrictions concerning the furnishing of samples apply.

The deposits were made according to the Budapest treaty on the international recognition of the deposit of microorganisms for the purposes of patent procedure at Deutsche Sammlung von Mikroorganismen und Zellkulturen GmbH (DSMZ), Inhoffenstr. 7B, D-38124 Braunschweig, Germany.

The Budapest Treaty provides that any restriction of public access to samples of deposited biological material must be irrevocably removed as of the date of grant of the relevant patent.

TABLE II
Deposited strains made at a depositary institution.

| Strain | Name | Accession no. | Deposit date |
|---|---|---|---|
| Bacillus | FHB-P | DSM34003 | 24 Aug. 2021 |
| amyloliquefaciens | | | |
| Bacillus | FHB-S31 | DSM34007 | 24 Aug. 2021 |
| amyloliquefaciens | | | |
| Bacillus | FHB-S4 | DSM34006 | 24 Aug. 2021 |
| amyloliquefaciens | | | |
| Bacillus | FHB-S21 | DSM34318 | 30 Jun. 2022 |
| amyloliquefaciens | | | |
| Bacillus | FHB-S11 | DSM34317 | 30 Jun. 2022 |
| amyloliquefaciens | | | |

1. A Bacillus amyloliquefaciens strain, or a mutant or variant thereof, with increased inhibitory effect on one or more pathogens compared to a parental strain of Bacillus amyloliquefaciens (FHB-P) deposited as DSM 34003 at Deutsche Sammlung von Mikroorganismen und Zellkulturen GmbH (DSMZ), Inhoffenstr. 7B, D-38124 Braunschweig, Germany, wherein the Bacillus amyloliquefaciens strain, or a mutant or variant thereof, is a derivative strain of FHB-P.

2. The Bacillus amyloliquefaciens strain according to claim 1, wherein said Bacillus amyloliquefaciens strain has increased production of one or more metabolites compared to said parental strain of Bacillus amyloliquefaciens, when cultured under the same conditions.

3. The Bacillus amyloliquefaciens strain according to claim 2, wherein said one or more metabolites are lipopeptides selected from the group consisting of iturins, fengycins and surfactins, and combinations thereof.

4. The Bacillus amyloliquefaciens strain according to claim 1, wherein said Bacillus amyloliquefaciens strain comprises one or more mutations compared to said parental strain of Bacillus amyloliquefaciens,
wherein said one or more mutations are located in one or more genes of said parental strain of Bacillus amyloliquefaciens selected from the group consisting of clpP, swrC, clpC, sigH, graR, ftsH, pdhC, degU, mcsB, rapI, and rapC, and combinations thereof.

5. The Bacillus amyloliquefaciens strain according to claim 1, wherein said one or more phytopathogens comprises Fusarium graminearum, Fusarium culmorum and/or Botrytis cinerea.

6. The Bacillus amyloliquefaciens strain according to claim 1, wherein said Bacillus amyloliquefaciens strain is selected from:
(i) the Bacillus amyloliquefaciens strain (FHB-S31) deposited as DSM.34007 at Deutsche Sammlung von Mikroorganismen und Zellkulturen GmbH (DSMZ), Inhoffenstr. 7B, D-38124 Braunschweig, Germany;
(ii) the Bacillus amyloliquefaciens strain (FHB-S4) deposited as DSM_34006 at Deutsche Sammlung von Mikroorganismen und Zellkulturen GmbH (DSMZ), Inhoffenstr. 7B, D-38124 Braunschweig, Germany;
(iii) the Bacillus amyloliquefaciens strain (FHB-S21) is deposited as DSM 34318 at Deutsche Sammlung von Mikroorganismen und Zellkulturen GmbH (DSMZ), Inhoffenstr. 7B, D-38124 Braunschweig, Germany; and
(iv) the Bacillus amyloliquefaciens strain (FHB-S11) is deposited as DSM 34317 at Deutsche Sammlung von Mikroorganismen und Zellkulturen GmbH (DSMZ), Inhoffenstr. 7B, D-38124 Braunschweig, Germany.

7. A method for preparing a Bacillus amyloliquefaciens strain comprising the steps of:
(i) providing a parental strain of Bacillus amyloliquefaciens;
(ii) subjecting said parental strain of Bacillus amyloliquefaciens to one or more mutagens to generate one or more Bacillus amyloliquefaciens progeny strains;
(iii) determining the inhibitory effect and/or production of one or more metabolites of said one or more Bacillus amyloliquefaciens progeny strains; and
(iv) selecting a Bacillus amyloliquefaciens progeny strain with increased inhibitory effect and/or increased production of one or more metabolites.

8. The method according to claim 7, wherein said one or more chemical mutagens are selected from ethyl methane sulphonate (EMS), alkylating agents (such as ethyl ethane sulphonate, methyl methane sulphonate, ethylene imines, etc.), base analogs, azides, and N-methyl-N′-nitro-N-nitroguanidine (MNNG), preferably EMS.

9. A Bacillus amyloliquefaciens strain obtainable by the method according to claim 1, wherein said parental Bacillus amyloliquefaciens strain is Bacillus amyloliquefaciens (FHB-P) deposited as DSM 34003 at Deutsche Sammlung von Mikroorganismen and Zellkulturen GmbH (DSMZ), Inhoffenstr, 7B, D-38124 Braunschweig, Germany.

10. A composition comprising:
(i) the Bacillus amyloliquefaciens strain according to claim 1; and/or
(ii) a fermentation product produced by the Bacillus amyloliquefaciens strain according to claim 1.

11. The composition according to claim 10, wherein said composition comprises:
(i) the Bacillus amyloliquefaciens strain, and
(ii) the fermentation product produced by the Bacillus amyloliquefaciens strain.

12. The composition according to claim 10, wherein said fermentation product comprises increased levels of one or more metabolites compared to a fermentation product produced by said parental strain of Bacillus amyloliquefaciens, when cultured under the same conditions.

13. A method of inhibiting growth of one or more phytopathogens on a plant, said method comprising applying a Bacillus amyloliquefaciens strain according to claim 1 to the plant or a part of the plant.

14. A method of inhibiting growth of one or more phytopathogens in a plant growth medium, said method comprising introducing the Bacillus amyloliquefaciens strain according to claim 1 into the plant growth medium.

15. A kit comprising:
(i) the Bacillus amyloliquefaciens strain according to claim 1;
(ii) a container.

16. An isolated Bacillus strain selected from:
(i) Bacillus amyloliquefaciens DSM 34007 and mutants and variants thereof;
(ii) Bacillus amyloliquefaciens DSM 34006 and mutants and variants thereof;
(iii) Bacillus amyloliquefaciens DSM 34318 and mutants and variants thereof; and
(iv) Bacillus amyloliquefaciens DSM 34317 and mutants and variants thereof.

17. A composition comprising the isolated Bacillus strain according to claim 16 in an agrochemically acceptable carrier.

18. A method comprising treating a plant and/or a plant propagation material and/or a plant growth medium with the isolated Bacillus strain according to claim 16.

19. A method comprising treating a plant and/or a plant propagation material and/or a plant growth medium with a fermentation product produced by the isolated Bacillus strain according to claim 16.


SEQUENCE LISTING (22 sequences)

SEQ ID NO: 1 - DNA, 597 bp, Bacillus amyloliquefaciens
  source 1..597: mol_type = genomic DNA
atgaatttaatacctacagtcattgaacaaacgaaccgcggagaaagagcttatgacatttactcccgtcttttgaaggaccgtattatcatgcttggatctgcgattgatgacaacgttgcaaactccatcgtgtcacagcttttattccttgaagcggaagaccctgaaaaagatatttccatttacattaacagccctggcggatcaatcactgccggtatggcaatctatgatacaatgcagttcattaaaccgaaagtatccacgatctgcatcggtatggccgcttctatgggagcattcttacttgctgccggagaaaaaggcaaacgttacgcgcttcctaacagtgaagtcatgattcaccagccgcttggcggagcgcaaggacaggcgactgaaattgaaatcgcggcaaaacgcattctcttgcttcgcgataaattaaacaaagtgcttgctgaacgtaccggccagccgcttgaagtgatcgagcgcgacacagaccgcgacaacttcaaatcagctgatgaagctttagaatacggcttaatcgataaagtgctgacacgcaatacggaagaccagaagtaa

SEQ ID NO: 2 - DNA, 3141 bp, Bacillus amyloliquefaciens
  source 1..3141: mol_type = genomic DNA
atgaaccacattattaactttgtactgaagaacaaattcgcagtttggctgatgacgattatcgttactgtagcagggctgtatgccggtctgaatatgaagcaggaatccataccggacgtcaacatgccttacctctcagtaaatacgacttatcccggagctgctccgagtcaagttgcggatgacgtgacgaaaccgatcgagcaggcggtgcagaatttagaaggagtgaacgtcgtatcgtccacatcctctgaaaacgtctcatcggttatgattgaatatgattacaacaaggatatggacaaggcgaaaaccgaagtcgctgaagcgctggataatgtcagccttcccgacgacgcgaaaaaacccgacatttcccgttacagcatcaactctttcccgattttgacactcagtgtgacaagcggcaaaagctcgcttgaagatctgaccaaaaacgtggaaaacactctcgttcctaagcttgaaggcattcagggcgttgcgtccgtgcaagtttccggacagcaggaagagcaggtggaattctctttcaaagataaaaaaatgaaagagtacggacttgatgaagatactgtcaagaaagtgatccaaggctctgacgtcaatacgccgcttggattatacacattcggcaacaaggaaaaatcagttgtcgttaacggaaatattacgtcaattaaagatctgaaagatatgagaattccggtcacatcttctgcagctcaaggtcaagcgggcggcgcgggtgcggcatctgcggcggacgctcaggctgcgcagcaggcacagcaaagcgcatcagcaggggtgccgacggttaagctttctgatattgccgacattaaagatgtgaaaaaagcagaatccatttcccgcacgaacggaaaggacagcatcggaattaatatcgtaaaagccaatgacgccaacacggtggaagtggccgatgcgattaaagatgaactgaatcaatacaaaaaagaccataaagggttcaagtacagctcgacactcgatatggccaagccgattacagagtcagtggatacgatgttaagcaaagcgattttcggcgcgatctttgcggtcgtgatcattctcttattcttaagagatattaaatcaacgatgatttctatcgtttccatcccgctgtcactgctgattgcgcttctcgtgctgaatcagcttgatgtcactttaaacattatgacgctcggtgcgatgacggtcgccatcggacgggtagtcgatgactccattgtcgttatcgaaaacatttaccgccgcatgaggctgaaagatgagcctttacgcggaaagcaattggtgcgtgaagcaacgaaagaaatgtttaaaccgatcatgtcatcaacgatcgtaaccattgccgtattcctgccgcttgccatggtcggcggacagatcggcgaactgtttatgccgtttgcattaacaatcgtctttgcgcttgccgcatcattgttgatttccatcacactggtgccaatgcttgcgcacagtttattcaaaaaatcgctcacaggcgcgcctgttaaagcgaaagagcataagccgggcagattggccgatttctataaaaaagtgctgaattggtcattaagccataaatggattacatctattatcgcggttctgatgcttgtcggcagtttattcctcgtaccgctgatcggcgcgagctatctgccggcgcaagcggacaaaacgatgcagctcacatatacgcctgaaccgggtgaaacgaaaagtgaagctgaaaaagctgcgcaaaaagcggaagacatgctgcttaaacgcaaacatgtcgataccgtccagtattcattaggctcgcaaagcccgctcggcggaagctcgaacggcgcattgttctacgtgaaatacgaagaagacacgcctgattttgacaaagaaaaagacaatgtcttaaaagaaatcaaaaaaacatccagccgcggcgaatggaaaacgcaaaatttcagttcgtcaggcaacaacaacgaattaacgtactatgtgtacggagattcagagtctgatattaaaggcacggtcaaagaaatcgaaagcatcatgaaaaagcagaaggatctgaaagacgtaaactcaggcctttccagcacatatgatgagtacacgttcgtcgctgaccaggagaagctcagcaagctcggcttaacggcttcgcaaatctcccaagccctgatgtcccaaacgtcacagtctcctttgacaacggtgaaaaaagacggcaaagagcttgacgtgaatatcaagactgaaaaagaccagtataagagtgtaaaagaattagaggataaaaccatcacatcgcctgcaggccaagaagtaaaaatcggcgatgtggcgaaagtgaaaaacggcacgacatctgacaccatttccagacgcgacggcaaagtgtacgctgacgtaacagcaaccgtcacttctgacaacatcacgaaggtgtcatcagccgttcaaaagaaagttgacaaactggataagccggacaatgtctccattgataccggcggtgtatcagcggatattgctgattcctttacgaaactcggcttagcgatgcttgcagcagtcgccattgtatatctcgttcttgtgatcacgttcggcggagcattagcgccgtttgcgattctgttctcgcttccgtttacggtcatcggcgcattagtcggactgtatgtatccggagagacagtcagcctgaatgccatgatcggcatgctgatgctgatcggaatcgtcgtcacaaatgcgattgtcttaattgaccgcgtcatccataaagaagcggaaggcttatcgacgagagaagcgctccttgaagccggatctacgagattgcgtccgattctgatgacggcgatcgcgacaatcggcgccctgcttccattagcattgggcttcgaaggcggaagccaggtcatttctaaaggactcggtgttaccgttatcggcggtttaatcagttcgactctgcttacccttctgattgtgccgatcgtatatgaagtattggtgaaattccgtaagaaaaaacctggaacggaagaagagtaa

SEQ ID NO: 3 - DNA, 2436 bp, Bacillus amyloliquefaciens
  source 1..2436: mol_type = genomic DNA
atgatgtttggaagatttacagagcgagctcaaaaggtattggcactggcacaggaagaagcactgcgcttaggccatacaaatatcggaactgaacatatattattggggttagtccgtgaaggtgaagggatagcatttaaagccttagaagcgctgggcctcaattcagataaaatgcaaaaagaagtggaaagtttgattggccgaggacaggaaagcaccacatctgtccctcattacacgcctcgagcaaaaaaggtaatagaactctcaatggatgaagccagaaagctgggacattcttatgtaggaacagaacacatacttctcgggctgattcgtgaaggcgagggagtcgccgcgagagttctgaataatctgggtgtcagtctcaataaggcgagacagcaggttctgcagcttctcggaagcaatgaaacagggacatctggagccgggacaaatagcagtgcaaacacgccgacgcttgacagccttgctcgtgatttaactgcgattgcgagggaagacagccttgatccggttatcggccgaagcaaagaaattcagcgtgttattgaggtattaagccgcagaacaaagaataaccccgttcttattggagaaccgggtgtaggtaaaactgcgattgccgaaggcctcgcacagcagatcatcaacaatgaagtgccggaaattttacgtgataaacgcgtaatgacgttagatatgggaacggttgtagccggcacgaaataccgcggagaatttgaagaccgcctgaaaaaggtaatggatgaaatacgtcaggccggcaatatcattttattcattgatgagctgcacacactgatcggagcgggaggagcagaaggtgcgattgacgcgtcgaatattttaaaaccttcactggcgcgcggagagcttcagtgcatcggtgcaacaacgcttgatgagtaccgcaaatatatcgaaaaagatgcagccctggagcgccgtttccagccgattcaggtggatgagccgtcagtcgatgaaagcattcaaattttaaaaggactccgtgaccgctatgaagcgcatcaccgcgtatccattaccgatgaagcgatcgaagcggcagttaaattgtccgaccgttatatatctgaccgcttccttccagataaagcgatcgatttaattgacgaagccggttcaaaagtgcgcctccgttctttcacaacgcctccgaacttaaaagagcttgagcagaagcttgatgaagttcgcaaggaaaaagacgctgccgttcaaagccaggaatttgaaaaagcggcttctcttcgtgatacggagcagcgcctgagagaacaggtggaagatacgaaaaaaacttggaaagaaaaacagggccaagagaactccgaagtttctgtagaggatatcgcgatggttgtatccagctggaccggggtgcctgtatctaaaattgcgcaaactgaaacagataagcttctcaatatggaaagcattctgcactcccgtgtcattggccaggatgaagccgttgtagccgttgcaaaggctgtgagacgtgcaagagccggtctgaaggatccgaaacgtccgattggttcattcatcttcttaggccctacaggcgtagggaagacagagctggcaagagcactggcggaatccattttcggtgatgaggaagcgatgatcagagtggatatgtccgaatacatggagaaacattcgacttcacgccttgtcggttctcctccgggatatgtcggctatgatgaaggcggccagctgacagaaaaagtgagaagaaaaccttactctgtcgtactgcttgatgaaattgaaaaagcgcatcctgatgtgtttaacattctcttgcaagtgcttgaagacggacgtttgactgattcaaaaggacgcactgttgatttccgcaacacgatcctgattatgacgtcaaacgtcggagcgagcgagctgaaacgcaacaaatatgtcggcttcaatgttcaggatgaatcacaaaatcataaagacatgaaagacaaagtgatgggagagctgaagcgtgccttcaggcctgagtttatcaaccggattgacgaaattatcgtcttccactcccttgagaaaaaacatcttacagacatcgtgtcgcttatgtctgaccagttaacaaaacgtctgaaagaacaagatctctctatagagctgacagaagctgcaaaagcaaaagtagcagacgagggcgtcgatttggaatacggcgcacgtccgttaagaagagcgatccaaaagcatgtggaggaccgtttatcagaagagctcctcagaggcaatattgataaagggcagcacatcgttcttgatgttgaggacggcgaatttgtcgtaaaaacaactgctaaaacgaactaa

SEQ ID NO: 4 - DNA, 657 bp, Bacillus amyloliquefaciens
  source 1..657: mol_type = genomic DNA
gtgaatctacagaacaacaagggaaaattcaacaaagtgcagttttgccagttggaggacgagcaggtcattgaaaaggttcatgttggggatagtgatgcgttagattacttgattacgaagtaccgtaattttgtacgggcgaaagccaggtcttattttttaataggggctgacagagaagatattgtacaggaagggatgataggcttatataagtctatccgagactttagagaggacaagctcacttcatttaaagcttttgcagaattatgcattacccgccagattattaccgctataaagacagctacccgtcagaaacacattcctttaaattcctatgtttctttagacaagcctatttttgatgaagaatcagacagaacactgcttgatgtcatctcaggcgctaaaacgttaaatcctgaagaaatgattattaatcaggaagaatttgatgatattgaaatgaaaatgggagaactgctgagcgatctggagagaaaggtgcttgttttgtacttagacggaagaagttaccaagaaatttcagatgacttaaaccgacacgtaaaatcaatcgataatgccttgcagcgtgtaaagcgcaagcttgagaagtatttagaaataagagaaatcagcatgtag

SEQ ID NO: 5 - DNA, 684 bp, Bacillus amyloliquefaciens
  source 1..684: mol_type = genomic DNA
ttggataatatatataaattgctgattatagaagacgataaggatatggctaatgcgttacaaaatttacttgttaaatggaattttgagactgttatatgcggagattttgataatatattgtcaatatatgaaaaagaagagcctcacatcatattattggacattaatattccttcatttgatggtttttattggtgtaagaaaataagagaaatttcaagtgtcccaattattttttgctcttctagaaacagtaatatggatattgtcatggctattaacaatggcggggatgattatattcaaaaaccatttgacagccatgtattggtggcgaaattacaagcaattataagaagaacttatgagtacataacagcagaacctcatttaatcgaatctaataatgtgatattaaaccttaacgatgcaaaaatatatttccaagatcagagtttagagcttacaaagaatgaatttaaaattctaaaaacgttaatggaaaacaagggaaaagttgtttcacgggataatttaatgagacagctttggaatgatgatatttacgtaaacgaaaatacgttgacagtaaatataaatagattaaggagtagattagaggggattaatgttgatgacttcattttaacaaaaaaaggaatgggatatataattcaatga

SEQ ID NO: 6 - DNA, 1920 bp, Bacillus amyloliquefaciens
  source 1..1920: mol_type = genomic DNA
atgaatcgggtcttccgtaataccattttttatttacttattttattagtagtaatcggggttgtgagttacttccagacttcaaacccgaaaacagaaaatatgtcgtacagtacgtttatcaagaatctcgatgaagggaaagttgacagcgtatcggttcagcctgtcagaggtgtttatgaggtaaaaggacagctgaaaaaatacgacaaagaccagtactttttaacacatgttcctgaaggaaaaggagcagaccagatatttgacgcaattaaaaagacggacgtaaaagttgatcccgcacaggaaacaagcgggtgggtgacgttcttaacgacgatcattccgtttgtcatcatctttattctgtttttcttcctgctcaatcaggctcagggcggcggaagccgtgtcatgaacttcggaaagagcaaagcgaagctgtatacggaagaaaagaaacgcgtgaagtttaaagatgtagccggcgcggatgaggagaagcaggagcttgttgaggttgttgagtttctgaaagatcctcgcaaatttgccgagctaggcgcgagaattcctaaaggcgtactgttggtcggacctccgggaaccggtaagacattgcttgcgagagcatcagccggtgaagcgggcgttcctttcttcagcatcagcggatctgacttcgttgaaatgtttgtcggtgtcggtgcttcccgtgttcgtgatttattcgaaaatgcgaaaaagaacgcgccctgcctgatcttcattgatgagatcgacgctgtgggacgtcagcgcggcgccggtcttggcggcgggcatgatgaacgcgaacagacgctgaaccagctgcttgtagagatggacggtttcagtgcgaatgaaggaatcattattatcgctgcgacaaaccgtgcggacattctggaccctgcattgctccgtccgggacgttttgaccgtcagattacggtagaccgtccggatgtcatcggccgtgaagccgtattgaaggtccatgcgagaaacaaaccgcttgatgagacggtgaacttaaaagcaattgcttctagaacacctggtttctcaggcgccgatttagaaaaccttcttaacgaagcggcactcgttgctgcccgtcaaaacaagaagaaaattgatatgcgcgacattgatgaagcaacggaccgtgtcattgcgggccctgcgaagaaaagccgcgtcatttcgaaaaaagagcgcaatatcgtggcgtaccacgaaggcggtcacaccgttatcggacttgtgctggatgaagccgatatggttcataaagtaacgatcgttccgagggggcaggcaggcggatacgcagtcatgcttccgagagaagaccgctacttccagacaaaacctgaacttctcgataaaattgtcggtcttctgggcggacgcgttgccgaggaaattatcttcggcgaagtcagcacgggggctcacaatgacttccagcgggcaacgaacattgccagaagaatggtgacggagttcggtatgtcagaaaaactcggaccgcttcagttcggtcaatctcaaggcggtcaagtattcctgggccgggatttcaacaacgagcaaaactacagtgatcagatcgcttacgaaatcgatcaggaaattcaacgcatcattaaagaatgttatgagcgtgcgaaaacaatcctgactgaaaatcgcgataagcttgaattgattgctcagacattgcttgatgtggaaacattagatgcggaacaaatcaagcatcttgtggatcacggaaaactcccagagcgcaatttctcagatgaccatcttcaaaaagacgatgtgaaagtaaacattctgtctaaaacagaagacgaaaagaaggacgaaccgaaagaatag

SEQ ID NO: 7 - DNA, 1194 bp, Bacillus amyloliquefaciens
  source 1..1194: mol_type = genomic DNA
atggctgtaaaagtagtaatgccgaaattgggaatggcgatgaagcaaggtgaagtatcagtctggaataagaaagtcggcgatccggttgaaaagggagaaagcatcgccagcatcaactctgaaaaaatagaaatggaagtagaagcgcccgaaagcggcacgctccttcacattaaagtcaaagagggagaaggagtcccgccgggaacggccatctgttatatcggggaaaacggtgaaaaggtgcaggagaaggaggcaccggcccctgaaaatgccggggaaccacaagcagaacctgaaactattcccgctcccaaagccgggaaagagcgtaaacaccgcgtgaaaatatcccctgtggcccgaaaaatggcggagaaggcaggattgaaagttgatacgctgaacgggacggggcccgggggcagaatcgtaaaagctgacgtcctaaaggcgatgaagacggaagctgaaccgcggtcttcagttcagacggcacagccggagggaaaaccggcctcagcaatgagaaaagtgattgcagaccgaatgcacaaaagcctgcaaaacagcgcccagctgacgctgaccatgaaagcggacattacggagcttgtgaaatggcagaaacagctggccgacagtgcgaaaaagcggagcggtgttaaactgacggtcacgcattttgtatcccgggccgcggtgcttgcgctgaaacagcatcctgagctgaacagctcttatcaagaggagcggatcattacctatccgcatgtccatctcgggatggcagtttctcttgaaaacggccttgtcgtgccggtgatccgtgatgcggaaaagctttcattccttgagcttgcggatcaaatcagcacatctgcacgccgggcgcgggaaggaaatgcttccggagatgatcttcacgggtcgactttttctattacaaatctcgggggatatggaattgaacatttcaccccgatccttaatccgcctgaagccggtattctcggagtgggcgcaagctatgaaacgccggcgttcaaaggagatgagcttgtcaaaagcaccgtgctgccgctgagcctcacctttgatcacagagtatgtgacggtgctccggctgcggacttcctgaaaacggttaaagcgctgcttgaagagccggccggattgattttatag

SEQ ID NO: 8 - DNA, 645 bp, Bacillus amyloliquefaciens
  source 1..645: mol_type = genomic DNA
gtgaatgtcatgatcgcagacgaccagtctattgtaagagaggggctgaaaatgatcctgagcctccacgaaggaattgaagtatcaggagaagcttcctgcggcgaagaagttcttcagttgcttccacaaaccgaaaccgatgtgattttgatggatatcagaatgcccggcatggacggaatcgaagcgacaaaagaggtaaaagccagatatccgtctgtaaaagtcatcattctgacaacgtttgaagatggacactacatttttgccgggctgaaaagcggtgctgacggttacatcttgaaggatgctgattcagatgaaattgtcgcctctcttcaagccgtttgtgaagggaatatgatgctgaaccccaaagtcacggttcaagtcgtaaaagcgctcaatcatatggacatgcctgccgtgcaggaggaaccgaaatcaaatctgacggagattctcacgccgagagagcttgatgtggcaaagcacattatggaaggcaaaagcaataaacgcattgccgcagacttgtttctgactgagggcaccgtgaagaactatgtatccagaatcctcgataaacttgaagtgtcaaacagaaccgagcttacattatatttgcaaaaaatgatataa

SEQ ID NO: 9 - DNA, 1092 bp, Bacillus amyloliquefaciens
  source 1..1092: mol_type = genomic DNA
atgtcgctcaagcattttattcaggacgcattaagcaattggatgaagcaagagggacctgaaagtgacattgtactcagcagccgtatacggttggcccgtaactttgaaaatataaagtttcctgcccggtatacgaatgaagaagcctcatccattattcagcaattcgaagatcatttttcaggaaaaaaactgcctgatatcggtcaatttcatttaatcagaatgagtgaagctcagcctctggaaaaacgggtgctgatggagaaacatctgatcagcccgaatttaacggaatcaccgtttggcggctgtctgctttctgaaaatgaggaagtcagcattatgttaaatgaagaagatcatatcagaattcaatgcctcttccccggcttccagcttctcaatgcgataaaagctgcaaaccaggttgatgactggattgaggagaaagtggattacgcctttggtgagaagagagggtacttaacaagctgtcctacaaatgtagggacggggctaagggcttcggtcatgatgcatttgccgggtctcgtattaactaggcaaatgaatcgaattataccggcaattaaccaattaggtttagttgttagaggaatttacggtgaaggcagcgaagcattaggaaatatctttcaaatttcaaatcaaatcacacttggaaagtcagaacaggacatcgtcgatgatttgaacagtgtggctgcccagctgattgagcaggaacgctcagcccgagaagcgctttatcaaacctcgaaaattgaattagaggaccgggtattcagatcttacggagttttatccaactgccgtatgattgaatcgaaggaaacggcgaaatgcctttctgatgttaggctggggatagacctggggatcattaaagggctatcaagcaatatacttaatgaactgatgattttgacacagccgggttttctccagcaatattcgggtggtgcattacggccgaacgaaagagacgtgagaagagcggctcttatcagggaaaggctgcatttagaaatgagtgcaaatagacaggaggatgattcgatatga

SEQ ID NO: 10 - DNA, 1194 bp, Bacillus amyloliquefaciens
  source 1..1194: mol_type = genomic DNA
ttgcaacaagttttcttgaacaaaggtaaaattccatatgaaattgttacaaaaaaattaaatgactggtatacagcgataaaaaataatcttgatatggaggcagtcaggttaaaaaatgaagttgaagaattaatgggacggatggagaagaatccagatgtgattttatatcatcagctattagatttccgacatgaaatgatgattagctaccttcaatctaaaaacgaacaagagttaaaaaatgcgtatgaagcactccaagaacatcaaggccagattaaagggatgttggattattacttctttttctttatgggaatgtacgatttcaaaagaaaggaattggtttctgctataagctcttatagaagagctgaacagtcactgaattatgttgatgatgagatcgaacatgcggagttctattataaaatggctgagatttattactatatgaagcaaacatatttttcattgaattacgttaagagagctatgcgtatttatgggaagtataaaaattatgaagatcgaatattaaggtgtaaatatataactgccggaaacttgattgattctttagattataatggggcgttagaaatatttaaagagacgttagaaatatcgaggaagataaagagacagcttcatgtatatacttccatgttgaatatggggattgtttataatcagattggagattttcagagggcttatgagtgtttaattgacgcctacaattactttgaaaaagaaaatcatgcgtgtattgagaagacccttttcaatttggctcatgttcaagcgagaaggggcgatttaaagtccgctgggtatttttatgaaaagggatattctgttgctgaagagaggaataattcagaatataaagcgaaattcgatctgttgaaggcaatttacctgactgatgggtgttataatctaattgataattcattttgtttctttagtacacataagatgtatggtgacattgaggaatttgggtttgaagttgcggaagtattcagaaaaaagggagatgcggaaaggtcatcgaagtactacaggcaagtgattttagccaaaaatgaaatccaaaaaggagagatgattagtgaaacgcaagttgatagccttgtcactagtagtaatgttgggattgagtatagcgggagtaaataa

SEQ ID NO: 11 - DNA, 1149 bp, Bacillus amyloliquefaciens
  source 1..1149: mol_type = genomic DNA
atgaagagcgggttaattccttcttcagcagtcgggcagaagattaacgagtggtaccgatacataagaacattcagcgtgccggatgcagagatcttgaaggccgagatcaaacaagagcttgaccatatggagcctgattcgaacttagtactttattattccctcatggaatttcgccatcagctgatgctggactatttggagccgctggagaaattgaaaatagaagaccagccgaccctgtcggaaattctcgataacatagataacagccaggccgggctcaaagggcttctcgattattacgtcaactttttcaaaggcatgtttgaatttgataaaagagaatttatatcagcgatcacgtattataaacaggctgagaaaaaactggccttcgtatcggaccacgtggagcgggcggagttttattttaaagtggccgaagcttattatcatatgaaacaaacgtatttttctttgatacatataaaaaatgcttatgaaatttacgtagagcaggacacatacaacgtccggatcattcaatgccattttgtgttcggcgtcaatttaatggatgaaagacgttttgaacaggcggccaaacattttcagcacgctctgcaaatggccgaaaaagaacaaaaagcgcagcttgtcgggagggcgctgtataacctcggactgtgctactacaatcagaaccgcatcgatgaagccataccgtactttgaacgggcagttgatacatttgaatcacaaagaatcgtcaattctctccctcaggcatattttctaatcacactcatttactttaaactcggaaaaaaagataaagcatcggaatatcacaagcggggatatgaatatgctaaagaaacagacgatcccgtttacacggtaaaattcgaatggctgcaggctctgtatcaggcgaaaccggatgaggaaaaaatcaacgcatgtttccgttacctagaagataaaaatttatacgccgatatagaggatttagcactagaagcagctaaatattactatgaacaaaattgctttaaaatttcttctgactattttctgaaagtcgaagaggcaaggaaacaaatacaaaggagtgagggtttgtatgaaattgaaatctaa

SEQ ID NO: 12 - protein, 198 aa, Bacillus amyloliquefaciens
MNLIPTVIEQTNRGERAYDIYSRLLKDRIIMLGSAIDDNVANSIVSQLLFLEAEDPEKDISIYINSPGGSITAGMAIYDTMQFIKPKVSTICIGMAASMGAFLLAAGEKGKRYALPNSEVMIHQPLGGAQGQATEIEIAAKRILLLRDKLNKVLAERTGQPLEVIERDTDRDNFKSADEALEYGLIDKVLTRNTEDQK

SEQ ID NO: 13 - protein, 1046 aa, Bacillus amyloliquefaciens
MNHIINFVLKNKFAVWLMTIIVTVAGLYAGLNMKQESIPDVNMPYLSVNTTYPGAAPSQVADDVTKPIEQAVQNLEGVNVVSSTSSENVSSVMIEYDYNKDMDKAKTEVAEALDNVSLPDDAKKPDISRYSINSFPILTLSVTSGKSSLEDLTKNVENTLVPKLEGIQGVASVQVSGQQEEQVEFSFKDKKMKEYGLDEDTVKKVIQGSDVNTPLGLYTFGNKEKSVVVNGNITSIKDLKDMRIPVTSSAAQGQAGGAGAASAADAQAAQQAQQSASAGVPTVKLSDIADIKDVKKAESISRTNGKDSIGINIVKANDANTVEVADAIKDELNQYKKDHKGFKYSSTLDMAKPITESVDTMLSKAIFGAIFAVVIILLFLRDIKSTMISIVSIPLSLLIALLVLNQLDVTLNIMTLGAMTVAIGRVVDDSIVVIENIYRRMRLKDEPLRGKQLVREATKEMFKPIMSSTIVTIAVFLPLAMVGGQIGELFMPFALTIVFALAASLLISITLVPMLAHSLFKKSLTGAPVKAKEHKPGRLADFYKKVLNWSLSHKWITSIIAVLMLVGSLFLVPLIGASYLPAQADKTMQLTYTPEPGETKSEAEKAAQKAEDMLLKRKHVDTVQYSLGSQSPLGGSSNGALFYVKYEEDTPDFDKEKDNVLKEIKKTSSRGEWKTQNFSSSGNNNELTYYVYGDSESDIKGTVKEIESIMKKQKDLKDVNSGLSSTYDEYTFVADQEKLSKLGLTASQISQALMSQTSQSPLTTVKKDGKELDVNIKTEKDQYKSVKELEDKTITSPAGQEVKIGDVAKVKNGTTSDTISRRDGKVYADVTATVTSDNITKVSSAVQKKVDKLDKPDNVSIDTGGVSADIADSFTKLGLAMLAAVAIVYLVLVITFGGALAPFAILFSLPFTVIGALVGLYVSGETVSLNAMIGMLMLIGIVVTNAIVLIDRVIHKEAEGLSTREALLEAGSTRLRPILMTAIATIGALLPLALGFEGGSQVISKGLGVTVIGGLISSTLLTLLIVPIVYEVLVKFRKKKPGTEEE

SEQ ID NO: 14 - protein, 811 aa, Bacillus amyloliquefaciens
MMFGRFTERAQKVLALAQEEALRLGHTNIGTEHILLGLVREGEGIAFKALEALGLNSDKMQKEVESLIGRGQESTTSVPHYTPRAKKVIELSMDEARKLGHSYVGTEHILLGLIREGEGVAARVLNNLGVSLNKARQQVLQLLGSNETGTSGAGTNSSANTPTLDSLARDLTAIAREDSLDPVIGRSKEIQRVIEVLSRRTKNNPVLIGEPGVGKTAIAEGLAQQIINNEVPEILRDKRVMTLDMGTVVAGTKYRGEFEDRLKKVMDEIRQAGNIILFIDELHTLIGAGGAEGAIDASNILKPSLARGELQCIGATTLDEYRKYIEKDAALERRFQPIQVDEPSVDESIQILKGLRDRYEAHHRVSITDEAIEAAVKLSDRYISDRFLPDKAIDLIDEAGSKVRLRSFTTPPNLKELEQKLDEVRKEKDAAVQSQEFEKAASLRDTEQRLREQVEDTKKTWKEKQGQENSEVSVEDIAMVVSSWTGVPVSKIAQTETDKLLNMESILHSRVIGQDEAVVAVAKAVRRARAGLKDPKRPIGSFIFLGPTGVGKTELARALAESIFGDEEAMIRVDMSEYMEKHSTSRLVGSPPGYVGYDEGGQLTEKVRRKPYSVVLLDEIEKAHPDVFNILLQVLEDGRLTDSKGRTVDFRNTILIMTSNVGASELKRNKYVGFNVQDESQNHKDMKDKVMGELKRAFRPEFINRIDEIIVFHSLEKKHLTDIVSLMSDQLTKRLKEQDLSIELTEAAKAKVADEGVDLEYGARPLRRAIQKHVEDRLSEELLRGNIDKGQHIVLDVEDGEFVVKTTAKTN

SEQ ID NO: 15 - protein, 218 aa, Bacillus amyloliquefaciens
MNLQNNKGKFNKVQFCQLEDEQVIEKVHVGDSDALDYLITKYRNFVRAKARSYFLIGADREDIVQEGMIGLYKSIRDFREDKLTSFKAFAELCITRQIITAIKTATRQKHIPLNSYVSLDKPIFDEESDRTLLDVISGAKTLNPEEMIINQEEFDDIEMKMGELLSDLERKVLVLYLDGRSYQEISDDLNRHVKSIDNALQRVKRKLEKYLEIREISM

SEQ ID NO: 16 - protein, 227 aa, Bacillus amyloliquefaciens
MDNIYKLLIIEDDKDMANALQNLLVKWNFETVICGDFDNILSIYEKEEPHIILLDINIPSFDGFYWCKKIREISSVPIIFCSSRNSNMDIVMAINNGGDDYIQKPFDSHVLVAKLQAIIRRTYEYITAEPHLIESNNVILNLNDAKIYFQDQSLELTKNEFKILKTLMENKGKVVSRDNLMRQLWNDDIYVNENTLTVNINRLRSRLEGINVDDFILTKKGMGYIIQ

SEQ ID NO: 17 - protein, 639 aa, Bacillus amyloliquefaciens
MNRVFRNTIFYLLILLVVIGVVSYFQTSNPKTENMSYSTFIKNLDEGKVDSVSVQPVRGVYEVKGQLKKYDKDQYFLTHVPEGKGADQIFDAIKKTDVKVDPAQETSGWVTFLTTIIPFVIIFILFFFLLNQAQGGGSRVMNFGKSKAKLYTEEKKRVKFKDVAGADEEKQELVEVVEFLKDPRKFAELGARIPKGVLLVGPPGTGKTLLARASAGEAGVPFFSISGSDFVEMFVGVGASRVRDLFENAKKNAPCLIFIDEIDAVGRQRGAGLGGGHDEREQTLNQLLVEMDGFSANEGIIIIAATNRADILDPALLRPGRFDRQITVDRPDVIGREAVLKVHARNKPLDETVNLKAIASRTPGFSGADLENLLNEAALVAARQNKKKIDMRDIDEATDRVIAGPAKKSRVISKKERNIVAYHEGGHTVIGLVLDEADMVHKVTIVPRGQAGGYAVMLPREDRYFQTKPELLDKIVGLLGGRVAEEIIFGEVSTGAHNDFQRATNIARRMVTEFGMSEKLGPLQFGQSQGGQVFLGRDFNNEQNYSDQIAYEIDQEIQRIIKECYERAKTILTENRDKLELIAQTLLDVETLDAEQIKHLVDHGKLPERNFSDDHLQKDDVKVNILSKTEDEKKDEPKE

SEQ ID NO: 18 - protein, 397 aa, Bacillus amyloliquefaciens
MAVKVVMPKLGMAMKQGEVSVWNKKVGDPVEKGESIASINSEKIEMEVEAPESGTLLHIKVKEGEGVPPGTAICYIGENGEKVQEKEAPAPENAGEPQAEPETIPAPKAGKERKHRVKISPVARKMAEKAGLKVDTLNGTGPGGRIVKADVLKAMKTEAEPRSSVQTAQPEGKPASAMRKVIADRMHKSLQNSAQLTLTMKADITELVKWQKQLADSAKKRSGVKLTVTHFVSRAAVLALKQHPELNSSYQEERIITYPHVHLGMAVSLENGLVVPVIRDAEKLSFLELADQISTSARRAREGNASGDDLHGSTFSITNLGGYGIEHFTPILNPPEAGILGVGASYETPAFKGDELVKSTVLPLSLTFDHRVCDGAPAADFLKTVKALLEEPAGLIL

SEQ ID NO: 19 - protein, 214 aa, Bacillus amyloliquefaciens
MNVMIADDQSIVREGLKMILSLHEGIEVSGEASCGEEVLQLLPQTETDVILMDIRMPGMDGIEATKEVKARYPSVKVIILTTFEDGHYIFAGLKSGADGYILKDADSDEIVASLQAVCEGNMMLNPKVTVQVVKALNHMDMPAVQEEPKSNLTEILTPRELDVAKHIMEGKSNKRIAADLFLTEGTVKNYVSRILDKLEVSNRTELTLYLQKMI

SEQ ID NO: 20 - protein, 363 aa, Bacillus amyloliquefaciens
MSLKHFIQDALSNWMKQEGPESDIVLSSRIRLARNFENIKFPARYTNEEASSIIQQFEDHFSGKKLPDIGQFHLIRMSEAQPLEKRVLMEKHLISPNLTESPFGGCLLSENEEVSIMLNEEDHIRIQCLFPGFQLLNAIKAANQVDDWIEEKVDYAFGEKRGYLTSCPTNVGTGLRASVMMHLPGLVLTRQMNRIIPAINQLGLVVRGIYGEGSEALGNIFQISNQITLGKSEQDIVDDLNSVAAQLIEQERSAREALYQTSKIELEDRVFRSYGVLSNCRMIESKETAKCLSDVRLGIDLGIIKGLSSNILNELMILTQPGFLQQYSGGALRPNERDVRRAALIRERLHLEMSANRQEDDSI

SEQ ID NO: 21 - protein, 397 aa, Bacillus amyloliquefaciens
MQQVFLNKGKIPYEIVTKKLNDWYTAIKNNLDMEAVRLKNEVEELMGRMEKNPDVILYHQLLDFRHEMMISYLQSKNEQELKNAYEALQEHQGQIKGMLDYYFFFFMGMYDFKRKELVSAISSYRRAEQSLNYVDDEIEHAEFYYKMAEIYYYMKQTYFSLNYVKRAMRIYGKYKNYEDRILRCKYITAGNLIDSLDYNGALEIFKETLEISRKIKRQLHVYTSMLNMGIVYNQIGDFQRAYECLIDAYNYFEKENHACIEKTLFNLAHVQARRGDLKSAGYFYEKGYSVAEERNNSEYKAKFDLLKAIYLTDGCYNLIDNSFCFFSTHKMYGDIEEFGFEVAEVFRKKGDAERSSKYYRQVILAKNEIQKGEMISETQVDSLVTSSNVGIEYSGSK

SEQ ID NO: 22 - protein, 382 aa, Bacillus amyloliquefaciens
MKSGLIPSSAVGQKINEWYRYIRTFSVPDAEILKAEIKQELDHMEPDSNLVLYYSLMEFRHQLMLDYLEPLEKLKIEDQPTLSEILDNIDNSQAGLKGLLDYYVNFFKGMFEFDKREFISAITYYKQAEKKLAFVSDHVERAEFYFKVAEAYYHMKQTYFSLIHIKNAYEIYVEQDTYNVRIIQCHFVFGVNLMDERRFEQAAKHFQHALQMAEKEQKAQLVGRALYNLGLCYYNQNRIDEAIPYFERAVDTFESQRIVNSLPQAYFLITLIYFKLGKKDKASEYHKRGYEYAKETDDPVYTVKFEWLQALYQAKPDEEKINACFRYLEDKNLYADIEDLALEAAKYYYEQNCFKISSDYFLKVEEARKQIQRSEGLYEIEI